(19) United States
(12) Patent Application Publication
(10) Pub. No.: US 2009/0082975 A1
(43) Pub. Date: Mar. 26, 2009

(54) METHOD OF SELECTING AN ACTIVE OLIGONUCLEOTIDE PREDICTIVE MODEL

(75) Inventors: Tamara Balac Sipes, San Diego, CA (US); Susan M. Freier, San Diego, CA (US); Kenneth Dobie, Del Mar, CA (US)

Correspondence Address: KNOBBE, MARTENS, OLSON & BEAR, LLP, 2040 MAIN STREET, FOURTEENTH FLOOR, IRVINE, CA, 92614, US

(73) Assignee: Isis Pharmaceuticals, Inc., Carlsbad, CA (US)

(21) Appl. No.: 12/237,330

(22) Filed: Sep. 24, 2008

Related U.S. Application Data
(63) Continuation of application No. 10/880,427, filed on Jun. 28, 2004, now abandoned.
(60) Provisional application No. 60/483,358, filed on Jun. 27, 2003.
(60) Provisional application No. 60/498,904, filed on Aug. 29, 2003.

(51) Int. Cl. G01N 33/48

(52) U.S. Cl. 702/20

(57) ABSTRACT

The present invention provides a method of identifying a predictor of antisense oligonucleotide activity by identifying properties of oligonucleotides, evaluating oligonucleotide activity of the oligonucleotides, and correlating oligonucleotide activity with the properties. A high correlation between oligonucleotide activity and a property indicates that the property is a predictor of oligonucleotide activity.

### CROSS REFERENCE TO ELATED APPLICATIONS

This application claims the benefit of U.S. Provisional Application No. 60/483,358, filed on Jun. 27, 2003; and U.S. Provisional Application No. 60/498,904, filed on Aug. 29, 2003. Each application is incorporated by reference herein in its entirety.

### BACKGROUND OF THE INVENTION

1. Field of the Invention

The present invention is relates generally to antisense oligonucleotide activity. In particular, the present invention is directed to a predictive model for selecting oligomers.

2. Description of the Related Art

Nucleic acid hybridization has been employed for investigating the identity and establishing the presence of nucleic acids. Hybridization is based on complementary base pairing. When complementary single stranded nucleic acids are incubated together, the complementary base sequences pair to form double-stranded hybrid molecules. The ability of single-stranded deoxyribonucleic acid (ssDNA) or ribonucleic acid (RNA) to form a hydrogen-bonded structure with a complementary nucleic acid sequence has been employed as an analytical tool in molecular biology research. The availability of radioactive nucleoside triphosphates of high specific activity and the development of methods for their incorporation into DNA and RNA has made it possible to identify, isolate, and characterize various nucleic acid sequences of biological interest. Nucleic acid hybridization has great potential in diagnosing disease states associated with unique nucleic acid sequences. These unique nucleic acid sequences may result from genetic or environmental change in DNA by insertions, deletions, inversions, point mutations, or by acquiring foreign DNA or RNA by means of infection by bacteria, molds, fungi, and viruses.

The mechanism of action for antisense oligonucleotides requires that the oligonucleotide hybridize to its mRNA target. Therefore, in principle, design of an antisense oligonucleotide requires that the oligonucleotide be complementary to the mRNA. In practice, when several oligonucleotides complementary to an mRNA are screened, certain antisense oligonucleotides are more active and more potent than others in suppressing specific gene expression. Alahari et al., Mot. Pharmacol, 1996, 50, 808-19; Bennett et al., J. Immunol, 1994, 152, 3530-40; Chiang et al., J. Biol. Chem., 1991, 266, 18162-71; Dean et al., J. Biol. Chem., 1994, 269, 16416-24; Dean et al., Biochem. Soc. Trans., 1996, 24, 623-9; Duff et al., J. Biol. Chem., 1995, 270, 7161-6; Lee et at, Shock, 1995, 4, 1-10; Lefebvre d'Hellencourt et al., Biochim. Biophys. Acta, 1996, 1317, 168-174; Miraglia et at., Int. J. Immunopharmacol., 1996, 18, 277-40; Stewart et al., Biochem. Pharmacol., 1996, 51, 461-9; Monia et al., Nat. Med. 1996, 2, 66875; Stepkowski et al., J. Immunot., 1995, 154, 1521, and J. Immunol., 1994, 153, 5336-46. In addition, some complementary oligonucleotides can show non-antisense effects. Ecker et al., Nuc. Acids Res., 1993, 21, 1853-6; Bennett et al., Nuc. Acids Res., 1994, 22, 3202-9; and Krieg et al., Nature, 1995, 374, 546-9. To date, the most effective approach for identifying oligonucleotides with good hybridization efficiency has been an empirical one. Such an approach involves the synthesis of large numbers of oligonucleotide probes for a given target nucleotide sequence. Arrays are formed that include the probes, and hybridization experiments determine which of the oligonucleotide probes exhibit good hybridization efficiencies. Examples of such an approach are found in D. Lockhart, et al., Nature Biotech., infra, L. Wodicka, et al., Nature Biotechnology, infra., and N. Milner, et al., Nature Biotech, infra. One major drawback to this approach is the vast number of oligonucleotides that must be synthesized in order to achieve a satisfactory result. Typically, about 2%-5% of the test probes synthesized yield acceptable signal levels.

The use of neural networks for oligonucleotide design has also been investigated. Neural networks are easily taught with real data; they therefore afford a general approach to many problems. However, their performance is limited by the training that they are given. In addition, a large amount of data is required to adequately teach a neural network to perform its job well A comprehensive database for either oligonucleotide array design or antisense suppression of gene expression has not been made available. For these reasons, the performance reported to date of neural network solutions against the probe design problem is mediocre.

Finally, approaches that have attempted to use target nucleic acid folding calculations to predict experimental results inferred to depend upon hybridization efficiency, e.g., antisense suppression of mRNA translation, have so far only demonstrated that the predictions of current nucleic acid folding calculations correlate poorly with observed behavior. The probable reason for this is that the structures predicted by such programs for long sequences are poor predictors of chemical reality; the results of experiments that attempt to confirm the predictions of such calculations support this assessment. Recent improvements to this approach, which use predicted RNA structure topology as a predictor of relative RNA/RNA association kinetics have been more successful at forecasting the results of antisense experiments. However, these methods are not computationally efficient, and have so far only been shown to work for targets of fewer than 100 bases in length. Such methods are therefore not yet capable of predicting the behavior of full-length mRNA targets, which are typically between 1,000 and 2,000 bases in length.

The most commonly used and most effective approach to discovery of antisense oligonucleotides involves synthesis of numerous oligonucleotides—typically up to several dozen—designed to hybridize to different regions of the targeted mRNA, followed by activity screening in cells. Bennett et al., Biochimica et Biophysics Acta, 1999, 1489, 19-30.

Several attempts have been made to identify features of oligonucleotides that are associated with antisense activity. Development of successful methods for selection of active oligonucleotides prior to oligonucleotide synthesis and cell-based screening would have two benefits. First, the cost of antisense discovery would be reduced and synthesis and screening of multiple compounds could be eliminated. Second, identification of the features associated with specific and non-specific effects of oligonucleotides would likely lead to a better understanding of the detailed mechanism of antisense activity and, potentially, to identification of compounds with even greater potency. Several groups have described combinatorial approaches for identification of optimal antisense sites in target mRNA using a cell free assay.

Typically, a library of randomized oligonucleotides is incubated with the target mRNA and RNAse H. Mapping of the most favored RNAse H cleavage sites results in identification of the most favored binding sites. This approach has been used to find sites for both antisense oligonucleotides (Ho et al., Nuc. Acids Res., 1996, 24, 1901-7; Ho et al., Nat. Biotechnol., 1998, 16, 59-63; Ho et al., Methods Enzymol., 2000, 314, 168-83; and Lima et al., J. Bicl. Chem., 1.997, 272, 626-38) and ribozymes (Birikh et al., RNA, 1997, 3, 429-37). It can, however, be complicated by interactions of library oligonucleotides with each other and by binding of multiple oligonucleotides to the mRNA target (Bruice et al., Biochemistry, 1997, 36, 5004-19). Concerns over library complexity have limited oligonucleotide lengths in these studies to 10 nucleotides (“nt”). Optimal binding sites for short oligonucleotides may not predict those for longer antisense oligonucleotides. Matveeva et al. (Nuc. Acids Res., 1997, 25, 5010-6) were able to use longer oligonucleotides and reduce library complexity by restricting the oligonucleotide pool to oligonucleotides complementary to the mRNA target sequence.

A similar but less thorough screen was performed by Jarvis et al. (J. Biol. Chem., 1996, 271, 29107-12) who used a cell free RNAse H assay with individual oligonucleotides to identify optimal sites for synthetic ribozymes. Optimal binding sites have also been identified without using RNAse H cleavage assays. Ecker et al. (Nuc. Acids Res., 1993, 21, 1853-6) screened randomized combinatorial libraries of 2′-O-methyl and phosphorothioate modified compounds and identified compounds that bind to H-ras mRNA. Using oligonucleotide arrays on glass slides, Southern and colleagues (Southern et al., Nuc. Acids Res., 1994, 22, 1368-73 and Milner et al., Nat. Biotechnol., 1997, 15, 53741) were able to identify compounds that bound tightly to c-raf mRNA and were able to select the site for ISIS 5132, the most potent c-raf antisense compound reported at that time. Their synthetic approach uses a strategy that results in synthesis of only oligonucleotides complementary to the mRNA of interest. The effectiveness of these cell-free approaches assumes that the most favored site(s) for oligonucleotide binding to the mRNA in the cell-free system will be the target site for the most active antisense oligonucleotide.

To test whether this was the case, Matveeva et al. (Matveeva, Nat. Biotechnol., 1998, 16, 1374-5) evaluated the correlation between activity in an RNAse H mapping assay or a gel shift binding assay with antisense activity in cells. Moderate correlation with cellular activity (R=0.6) was found for both cell-free assays. Similar correlation analysis of the randomized library data of Ho (Ho et al., Nuc. Acids Res., 1996, 24, 1901-7 and Ho et al., Nat. Biotechnol., 1998,16, 59-63) and the array data of Mir (Southern et al., Ciba found. Symp., 1997, 209, 38-44) gave coefficients of correlation between activity in the cell free assay and antisense activity ranging from 0.2-0.7. Thus, the correlation between activity in the cell-free assay and antisense activity is relatively weak.

In spite of the relatively weak correlation observed between oligonucleotide binding in the cell free assay and antisense activity, ribozymes (Birikh et al., RNA, 1997, 3, 429-37) or antisense (Ho et al., Nuc. Acids Res., 1996, 24, 1901-7; Ho et al., Nat. Biotechnol., 1998,16, 59-63; Lima et al., Biol. Chem., 1997, 272, 626-38; and Matveeva et al., Nuc. Acids Res., 1997, 25, 5016-6) designed to sites identified by combinatorial selection were more likely to be active than those selected without initial cell-free screening. Thus, these methods can improve the “hit rate” for antisense discovery. However, these methods are cumbersome and, at best, result in several leads that still need to be screened in a cell-based assay. Therefore the benefit of improved hit rate may not make up for the substantial cost disadvantage associated with these cell free combinatorial assays.

Computational predictions of hybridization affinity that take into account RNA target structure, oligonucleotide self structure and oligonucleotide-RNA hybridization have had limited success at identifying potent antisense sites. Previous work (Tu et al, 1998, Matveeva et al, 2000, Giddings et al, 2002) has revealed a correlation between the short sequence motifs (tetramotifs or shorter) and antisense oligonucleotide activity. Separately, researchers also identified a correlation of certain ΔG energy values and oligonucleotide activity.

Further building on previous work includes both the ΔG energies and motifs, as well as other descriptors to help build a more efficient predictive model of oligonucleotide activity. Other features include oligonucleotide base information (oligonucleotide sequence information, A, C, T and G content), cell line information and concentration values. Cell-based screening of a number of compounds is still required. Combinatorial approaches offer the potential of finding the best antisense oligonucleotide for any target. These approaches have not, in general, identified compounds with substantially greater activity than those designed by more conventional methods. In addition, significant effort is required for the cell-free screen and several compounds must still be screened in cell-based assays. Although no single approach has yet provided a method for identifying the single best target site for an antisense oligonucleotide, several guidelines have been identified that may improve “hit rates” and avoid screening of compounds likely to have non-antisense activities. Thus, there continues to be a need for improved methods of predicting oligonucleotide activity.

### SUMMARY OF THE INVENTION

The present invention enables an improved method of predicting oligonucleotide activity. In one embodiment, the present invention provides a method of selecting a preferred set of oligomers from a large collection of oligomers such as a library of oligomers. The method involves choosing of a selection paradigm or selection algorithm to be used as a predictor of oligo activity based on the selected target and properties and attributes of the oligo. A method of this embodiment further involves choosing another selection paradigm to apply against the group, or set of oligos. The result of these two steps is two groups of selected oligos having predicted activity. A third selection paradigm or algorithm is then applied against or to the combined grouping of the first two selected oligos providing thereby a third, most select group of oligos having predicted activity according to the chosen selection paradigms or algorithms. In one embodiment the first selection paradigm, the second selection paradigm and the third selection paradigm are the same; in another embodiment, they are independently determined. The selection paradigms may be selected from the group consisting of decision tree, neural network, hierarchical clustering, clustering, regression tree, and combinations thereof.

The present invention also includes a database schema for a database of oligomers and related indicia forming a decision tree predictive model. The database stores and correlates a plurality of attributes for a plurality of oligomers, including a flex-motif, an RNAse H motif, an amplicon, a feature, a sequence, an energy, a structure, an oligomer activity and a cell line. The database further includes an influence indicator, providing an indication of the quantum of influence the attribute exerts on an oligomer activity. The database also preferably includes an activity manipulator for modulating the influence indicator according to the influence of the oligomer attributes on the oligomer activity.

The present invention also includes a system for designing a set of potentially active oligomers having at least a threshold level of predicted activity against a target, according to at least one design paradigm.

The present invention also provides a method of selecting a set of active oligomers using a combination of more than one selection paradigms by intersecting the results of oligomer selection according to selection algorithms and where the combination is synergistic.

The present invention also enables a method of designing a potentially active oligomer for a target nucleic acid by determining a set of defining design attributes according to at least one design paradigm, a total nucleotide length for the potentially active oligomer and a threshold level of predicted activity for the potentially active oligomer; combining a first and a second nucleotide according to the paradigm, thereby providing a first subset of the potentially active oligomer; and using an activity predicting system to predict activity of the first subset of the potentially active oligomer against the target; and repeating these steps so long as the predictive activity remains at least equal to the threshold value and the number of combined nucleotides in the first subset is less then the total nucleotide length.

The present invention further provides methods of identifying a predictor of antisense oligonucleotide activity by identifying a plurality of properties for a plurality of oligonucleotides. The present invention further provides methods for selecting a predictive paradigm for an application of interest; evaluating oligonucleotide activity of a plurality of oligonucleotides; and correlating oligonucleotide activity for a plurality of oligonucleotides with the plurality of properties. A high correlation between oligonucleotide activity and a property indicates that the property is a predictor of antisense oligonucleotide activity.

In one embodiment, properties include hybridization position of an oligonucleotide to its target; thermodynamics, number of nucleotide bases, proximity of binding to secondary structure of the target, presence of oligonucleotide sequence motifs, pyrimidine content, A+T content, presence of RNAse cleavage sites, RNAseH activity, target binding affinity, target specificity, isoform specificity, crosspieces activity, cleavage products and oligonucleotide chemistry. In one embodiment oligonucleotide activity includes modulation of protein synthesis, modulation of mRNA, modulation of protein activity, and modulation of cell viability.

The present invention is also directed to methods of identifying a predictor of antisense oligonucleotide activity by determining oligonucleotide target regions using feature-based or homology-based parameters, preparing oligonucleotides directed to target regions, identifying a plurality of properties for a plurality of oligonucleotides, evaluating oligonucleotide activity for a plurality of oligonucleotides, ranking oligonucleotides in a hierarchy of oligonucleotide activity, and correlating oligonucleotide activity for a plurality of oligonucleotides with the plurality of properties. A highly ranked oligonucleotide preferably includes a high correlation between oligonucleotide activity and a property, wherein the property is a predictor of antisense oligonucleotide activity. In one embodiment, the hierarchy is optimized to allow complex combinations of properties.

The present invention is also directed to methods of enhancing identification of an active oligonucleotide by eliminating at least the bottom five percent of oligonucleotides in the hierarchy or selecting at least one oligonucleotide from the top five percent of oligonucleotides in the hierarchy.

The present invention is also directed to methods for evaluating multiple predictive paradigms useful in predicting oligonucleotides having at least a baseline activity against a target. This aspect further facilitates the selection of a predictive algorithm according to the desired outcome and/or philosophical perspective on predictive factors.

### BRIEF DESCRIPTION OF THE DRAWINGS

FIG. 1 illustrates a block diagram of a system for predicting oligonucleotide activity in accordance with an embodiment of the present invention.

FIG. 2 is a diagram of an architecture of a hybrid predictive model in accordance with an embodiment of the present invention.

These figures depict a preferred embodiment of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE PREFERRED EMBODIMENTS

### Definitions

Before proceeding further with a description of the specific embodiments of the present invention, a number of terms will be defined.

Nucleic Acids

Polynucleotide—a compound or composition that is a polymeric nucleotide or nucleic acid polymer. The polynucleotide may be a natural compound or a synthetic compound. In the context of an assay, the polynucleotide is often referred to as a polynucleotide analyte. The polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. Isolation of a polynucleotide from the natural state often results in fragmentation. The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, the genomes of biological material such as microorganisms, e.g., bacteria, yeasts, viruses, viroids, molds, fungi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, cDNA, and the like. The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site specific chemical cleavage method. For purposes of this invention, the polynucleotide, or a cleaved fragment obtained from the polynucleotide, will usually be at least partially denatured or single stranded or treated to render it denatured or single stranded. Such treatments are well known in the art and include, for instance, heat or alkali treatment, or enzymatic digestion of one strand. For example, dsDNA can be heated at 90-100.degree. C. for a period of about 1 to 10 minutes to produce denatured material.

Target nucleotide sequence—a sequence of nucleotides to be identified, usually existing within a portion or all of a polynucleotide, usually a polynucleotide analyte. The identity of the target nucleotide sequence generally is known to an extent sufficient to allow preparation of various sequences that hybridizable with the target nucleotide sequence and of oligonucleotides, such as probes and primers, and other molecules necessary for conducting methods in accordance with the present invention, an amplification of the target polynucleotide, and so forth. The target sequence usually contains from about 30 to 5,000 or more nucleotides, preferably 50 to 1,000 nucleotides. The target nucleotide sequence is generally a fraction of a larger molecule or it may be substantially the entire molecule such as a polynucleotide as described above. The minimum number of nucleotides in the target nucleotide sequence is selected to assure that the presence of a target polynucleotide in a sample is a specific indicator of the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g. transcription of a DNA template into RNA) and the efficiency of detection and/or amplification of the target nucleotide sequence, where appropriate.

Oligonucleotide—a polynucleotide, usually single stranded, usually a synthetic polynucleotide but may be a naturally occurring polynucleotide. The oligonucleotide(s) are usually comprised of a sequence of at least 5 nucleotides, preferably, 10 to 100 nucleotides, more preferably, 20 to 50 nucleotides, and usually 10 to 30 nucleotides, more preferably, 20 to 30 nucleotides, and desirably about 25 nucleotides in length. Various techniques can be employed for preparing an oligonucleotide. Such oligonucleotides can be obtained by biological synthesis or by chemical synthesis. For short sequences (up to about 100 nucleotides), chemical synthesis will frequently be more economical as compared to the biological synthesis. In addition to economy, chemical synthesis provides a convenient way of incorporating low molecular weight compounds and/or modified bases during specific synthesis steps. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. For longer sequences standard replication methods employed in molecular biology can be used such as the use of M13 for single stranded DNA as described by J. Messing (1983) Methods Enzymol, 101:20-78. Other methods of oligonucleotide synthesis include phosphotriester and phosphodiester methods (Narang, et al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al., Proc. Nat. Acad. Sci. USA (1994) 91:5022-5026.

Oligonucleotide probe—an oligonucleotide employed to bind to a portion of a polynucleotide such as another oligonucleotide or a target nucleotide sequence. The design and preparation of the oligonucleotide probes are generally dependent upon the sensitivity and specificity required, the sequence of the target polynucleotide and, in certain cases, the biological significance of certain portions of the target polynucleotide sequence.

Oligonucleotide primer(s)—an oligonucleotide that is usually employed in a chain extension on a polynucleotide template such as in, for example, an amplification of a nucleic acid. The oligonucleotide primer is usually a synthetic nucleotide that is single stranded, containing a sequence at its 3′-end that is capable of hybridizing with a defined sequence of the target polynucleotide. Normally, an oligonucleotide primer has at least 80%, preferably 90%, more preferably 95%, most preferably 100%, complementarity to a defined sequence or primer binding site. The number of nucleotides in the hybridizable sequence of an oligonucleotide primer should be such that stringency conditions used to hybridize the oligonucleotide primer will prevent excessive random non-specific hybridization. Usually, the number of nucleotides in the oligonucleotide primer will be at least as great as the defined sequence of the target polynucleotide, namely, at least ten nucleotides, preferably at least 15 nucleotides, and generally from about 10 to 200, preferably 20 to 50, nucleotides. In general, in primer extension, amplification primers hybridize to, and are extended along (chain extended), at least the target nucleotide sequence within the target polynucleotide and, thus, the target sequence acts as a template. The extended primers are chain “extension products.” The target sequence usually lies between two defined sequences but need not. In general, the primers hybridize with the defined sequences or with at least a portion of such target polynucleotide, usually at least a ten-nucleotide segment at the 3′-end thereof and preferably at least 15, frequently a 20 to 50 nucleotide segment thereof.

Nucleoside triphosphates—nucleosides having a 5′-triphosphate substituent. The nucleosides are pentose sugar derivatives of nitrogenous bases of either purine or pyrimidine derivation, covalently bonded to the 1′-Carbon of the pentose sugar, which is usually a deoxyribose or a ribose. The purine bases include adenine (A), guanine (G), inosine (I), and derivatives and analogs thereof. The pyrimidine bases include cytosine (C), thymine (T), uracil (U), and derivatives and analogs thereof. Nucleoside triphosphates include deoxyribonucleoside triphosphates such as the four common deoxyribonucleoside triphosphates dATP, dCTP, dGTP and dTTP and ribonucleoside triphosphates such as the four common triphosphates rATP, rCTP, rGTP and rUTP. The term “nucleoside triphosphates” also includes derivatives and analogs thereof, which are exemplified by those derivatives that are recognized and polymerized in a similar manner to the underivatized nucleoside triphosphates.

Nucleotide—a base-sugar-phosphate combination that is the monomeric unit of nucleic acid polymers, i.e., DNA and RNA. The term “nucleotide” as used herein includes modified nucleotides as defined below.

DNA—deoxyribonucleic acid.

RNA—ribonucleic acid.

Modified nucleotide—a unit in a nucleic acid polymer that contains a modified base, sugar or phosphate group. The modified nucleotide can be produced by a chemical modification of the nucleotide either as part of the nucleic acid polymer or prior to the incorporation of the modified nucleotide into the nucleic acid polymer. For example, the methods mentioned above for the synthesis of an oligonucleotide may be employed. In another approach a modified nucleotide can be produced by incorporating a modified nucleoside triphosphate into the polymer chain during an amplification reaction. Examples of modified nucleotides, by way of illustration and not limitation, include dideoxynucleotides, derivatives or analogs that are biotinylated, amine modified, alkylated, fluorophore-labeled, and the like and also include phosphorothioate, phosphite, ring atom modified derivatives, and so forth.

Nucleoside—is a base-sugar combination or a nucleotide lacking a phosphate moiety.

Nucleotide polymerase—a catalyst, usually an enzyme, for forming an extension of a polynucleotide along a DNA or RNA template where the extension is complementary thereto. The nucleotide polymerase is a template dependent polynucleotide polymerase and utilizes nucleoside triphosphates as building blocks for extending the 3′-end of a polynucleotide to provide a sequence complementary with the polynucleotide template. Usually, the catalysts are enzymes, such as DNA polymerases, for example, prokaryotic DNA polymerase (1, II, or III), T4 DNA polymerase, T7 DNA polymerase, Klenow fragment, reverse transcriptase, Vent DNA polymerase, Pfu DNA polymerase, Taq DNA polymerase, and the like, or RNA polymerases, such as T3 and T7 RNA polymerases. Polymerase enzymes may be derived from any source such as cells, bacteria such as E. coli, plants, animals, virus, thermophilic bacteria, and so forth.

Amplification of nucleic acids or polynucleotides—any method that results in the formation of one or more copies of a nucleic acid or polynucleotide molecule (exponential amplification) or in the formation of one or more copies of only the complement of a nucleic acid or polynucleotide molecule (linear amplification).

Hybridization (hybridizing) and binding—in the context of nucleotide sequences these terms are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like.

Hybridization efficiency—the productivity of a hybridization reaction, measured as either the absolute or relative yield of oligonucleotide probe/polynucleotide target duplex formed under a given set of conditions in a given amount of time.

Homologous or substantially identical polynucleotides—In general, two polynucleotide sequences that are identical or can each hybridize to the same polynucleotide sequence are homologous. The two sequences are homologous or substantially identical where the sequences each have at least 90%, preferably 100%, of the same or analogous base sequence where thymine (T) and uracil (U) are considered the same. Thus, the ribonucleotides A, U, C and G are taken as analogous to the deoxynucleotides dA, dT, dC, and dG, respectively. Homologous sequences can both be DNA or one can be DNA and the other RNA.

Complementary—Two sequences are complementary when the sequence of one can bind to the sequence of the other in an anti-parallel sense wherein the 3′-end of each sequence binds to the 5′-end of the other sequence and each A, T(U), C, and C of one sequence is then aligned with a T(U), A, C, and G, respectively, of the other sequence. RNA sequences can also include complementary G/U or U/G base pairs.

Member of a specific binding pair (“sbp member”)—one of two different molecules, having an area on the surface or in a cavity that specifically binds to and is thereby defined as complementary with a particular spatial and polar organization of the other molecule. The members of the specific binding pair are referred to as cognates or as ligand and receptor (antiligand). These may be members of an immunological pair such as antigen-antibody, or may be operator-repressor, nuclease-nucleotide, biotin-avidin, hormones-hormone receptors, nucleic acid duplexes, IgG-protein A, DNA-DNA, DNA-RNA, and the like.

Ligand—any compound for which a receptor naturally exists or can be prepared.

Receptor (“antiligand”)—any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, repressors, protection enzymes, protein A, complement component Clq, DNA binding proteins or ligands and the like.

Oligonucleotide Properties

Potential of an oligonucleotide to hybridize—the combination of duplex formation rate and duplex dissociation rate that determines the amount of duplex nucleic acid hybrid that will form under a given set of experimental conditions in a given amount of time.

Parameter—a factor that provides information about the hybridization of an oligonucleotide with a target nucleotide sequence. Generally, the factor is one that is predictive of the ability of an oligonucleotide to hybridize with a target nucleotide sequence. Such factors include composition factors, thermodynamic factors, chemosynthetic efficiencies, kinetic factors, and the like.

Parameter predictive of the ability to hybridize—a parameter calculated from a set of oligonucleotide sequences wherein the parameter positively correlates with observed hybridization efficiencies of those sequences. The parameter is, therefore, predictive of the ability of those sequences to hybridize. “Positive correlation” can be rigorously defined in statistical terms. The correlation coefficient ρ_{x,y} of two experimentally measured discreet quantities x and y (N values in each set) is defined as

$${ρ}_{x , y} = \frac{Covariance ( x , y )}{\sqrt{Variance ( x ) Variance ( y )}}$$

where the Covariance (x,y) is defined by

$$Covariance ( x , y ) = \frac{1}{N} {\sum }_{j = 1}^{N} ( {x}_{j} - {u}_{x} ) ( {y}_{j} - {u}_{j} )$$

The quantities μₓ and μ_{y} are the averages of the quantities x and y, while the variances are simply the squares of the standard deviations (defined below). The correlation coefficient is a dimensionless (unitless) quantity between −1 and 1. A correlation coefficient of 1 or −1 indicates that x and y have a linear relationship with a positive or negative slope, respectively. A correlation coefficient of zero indicates no relationship; for example, two sets of random numbers will yield a correlation coefficient near zero. Intermediate correlation coefficients indicate intermediate degrees of relatedness between two sets of numbers. The correlation coefficient is a good statistical measure of the degree to which one set of numbers predicts a second set of numbers.

Composition factor—a numerical factor based solely on the composition or sequence of an oligonucleotide without involving additional parameters, such as experimentally measured nearest-neighbor thermodynamic parameters. For instance, the fraction (G+C), given by the formula

$${f}_{G , C} = \frac{{n}_{G} + {n}_{C}}{{n}_{G} + {n}_{C} + {n}_{A} + {n}_{TorU}}$$

where n_{G}, n_{C}, n_{A} and n_{T or U} are the numbers of G, C, A and T (or U) bases in an oligonucleotide, is an example of a composition factor. Examples of composition factors, by way of illustration and not limitation, are mole fraction (G+C), percent (G+C), sequence complexity, sequence information content, frequency of occurrence of specific oligonucleotide sequences in a sequence database and so forth.

Thermodynamic factor—numerical factors that predict the behavior of an oligonucleotide in some process that has reached equilibrium. For instance, the free energy of duplex formation between an oligonucleotide and its complement is a thermodynamic factor. Thermodynamic factors for systems that can be subdivided into constituent parts are often estimated by summing contributions from the constituent parts. Such an approach is used to calculate the thermodynamic properties of oligonucleotides Examples of thermodynamic factors, by way of illustration and not limitation, are predicted duplex melting temperature, predicted enthalpy of duplex formation, predicted entropy of duplex formation, free energy of duplex formation, predicted melting temperature of the most stable intramolecular structure of the oligonucleotide or its complement, predicted enthalpy of the most stable intramolecular structure of the oligonucleotide or its complement, predicted entropy of the most stable intramolecular structure of the oligonucleotide or its complement, predicted free energy of the most stable intramolecular structure of the oligonucleotide or its complement, predicted melting temperature of the most stable hairpin structure of the oligonucleotide or its complement, predicted enthalpy of the most stable hairpin structure of the oligonucleotide or its complement, predicted entropy of the most stable hairpin structure of the oligonucleotide or its complement, predicted free energy of the most stable hairpin structure of the oligonucleotide or its complement, thermodynamic partition function for intramolecular structure of the oligonucleotide or its complement and the like.

Chemosynthetic efficiency—oligonucleotides and nucleotide sequences may both be made by sequential polymerization of the constituent nucleotides. However, the individual addition steps are not perfect; they instead proceed with some fractional efficiency that is less than unity. This may vary as a function of position in the sequence. Therefore, what is really produced is a family of molecules that consists of the desired molecule plus many truncated sequences. These “failure sequences” affect the observed efficiency of hybridization between an oligonucleotide and its complementary target. Examples of chemosynthetic efficiency factors, by way of illustration and not limitation, are coupling efficiencies, overall efficiencies of the synthesis of a target nucleotide sequence or an oligonucleotide probe, and so forth.

Kinetic factor—numerical factors that predict the rate at which an oligonucleotide hybridizes to its complementary sequence or the rate at which the hybridized sequence dissociates from its complement are called kinetic factors. Examples of kinetic factors are steric factors calculated via molecular modeling or measured experimentally, rate constants calculated via molecular dynamics simulations, associative rate constants, dissociative rate constants, enthalpies of activation, entropies of activation, free energies of activation, and the like.

Predicted duplex melting temperature—the temperature at which an oligonucleotide mixed with a hybridizable nucleotide sequence is predicted to form a duplex structure (double-helix hybrid) with 50% of the hybridizable sequence. At higher temperatures, the amount of duplex is less than 50%; at lower temperatures, the amount of duplex is greater than 50%. The melting temperature Tₘ (° C.) is calculated from the enthalpy (ΔH), entropy (ΔS) and C, the concentration of the most abundant duplex component (for hybridization arrays, the soluble hybridization target), using the equation

$${T}_{m} = \frac{Δ H}{Δ S + R ln C} - 273.5$$

where R is the gas constant, 1.987 cal/(mole-° K). For longer sequences (>100 nucleotides), Tₘ can also be estimated from the mole fraction (G+C), ·χ_{G+C}, using the equation

Tₘ=81.5+41.0χ_{G+C}

Melting temperature corrected for salt concentration—polynucleotide duplex melting temperatures are calculated with the assumption that the concentration of sodium ion, Na⁺, is 1 M. Melting temperatures T′ₘ calculated for duplexes formed at different salt concentrations are corrected via the semi-empirical equation

T′ₘ([Na⁺])=Tₘ+16.6 log([Na⁺]).

Predicted enthalpy, entropy and free energy of duplex formation—the enthalpy (ΔH), entropy and free energy (ΔG) are thermodynamic state functions, related by the equation ΔG=ΔH−T ΔS, where T is the temperature in ° K. In practice, the enthalpy and entropy are predicted via a thermodynamic model of duplex formation (the “nearest neighbor” model which is explained in more detail below), and used to calculate the free energy and melting temperature.

Predicted free energy of the most stable intramolecular structure of an oligonucleotide or its complement—single-stranded DNA and RNA molecules that contain self-complementary sequences can form intramolecular secondary structures. For any given oligonucleotide there are at least two secondary structure. One where the oligo base pairs with itself forming a low energy hairpin structure. The second major structure is amorphous and is determined by numerous factors. This second structure may, for instance include structures such as stem loops, bulges, pseudo knots, knots, bulge-loops and others as discussed elsewhere, and as known in the art. For either type of structure, a value of the free energy of that structure can be calculated, relative to the unpaired strand, by means of a thermodynamic model similar to that used to calculate the free energy of a base-paired duplex structure. Again, the free energy ΔG is calculated from the enthalpy ΔH and the entropy ΔS at a given absolute temperature T via the equation ΔG=ΔH−TΔS. However, in this case there is the added difficulty that the lowest energy structure must be found. For a simple hairpin structure, this optimization can be performed via a relatively simple search algorithm. For more complex structures (such as a cloverleaf a dynamic programming algorithm, such as that implemented in the program MFOLD, must be used.

Coupling efficiencies—chemosynthetic efficiencies are called coupling efficiencies when the synthetic scheme involves successive attachment of different monomers to a growing oligomer; a good example is oligonucleotide synthesis via phosphoramidite coupling chemistry.

Algorithmic Operations:

Evaluating a parameter—determination of the numerical value of a numerical descriptor of a property of an oligonucleotide sequence by means of a formula, algorithm or look-up table.

Filter—a mathematical rule or formula that divides a set of numbers into two subsets. Generally, one subset is retained for further analysis while the other is discarded. If the division into two subsets is achieved by testing the numbers against a simple inequality, then the filter is referred to as a “cut-off”. In the context of the current invention, an example by way of illustration and not limitation is the statement “The predicted self structure free energy must be greater than or equal to −0.4 kcal/mole,” which can be used as a filter for oligonucleotide sequences; this particular filter is also an example of a cut-off.

Filter set—A set of rules or formulae that successively winnow a set of numbers by identifying and discarding subsets that do not meet specific criteria. In the context of the current invention, an example by way of illustration and not limitation is the compound statement “the predicted self structure free energy must be greater than or equal to −0.4 kcal/mole and the predicted RNA/DNA heteroduplex melting temperature must lie between 600° C. and 85° C.,” which can be used as a filter set for oligonucleotide sequences.

Examining a parameter—comparing the numerical value of a parameter to some cutoff-value or filter.

Statistical sampling of a cluster—extraction of a subset of oligonucleotides from a cluster of oligonucleotides based upon some statistical measure, such as rank by oligonucleotide starting position in the sequence complementary to the target sequence.

First quartile, median and third quartile—If a set of numbers is ranked by value, then the value that divides the lower ¼ from the upper ¾ of the set is the first quartile, the value that divides the set in half is the median and the value that divides the lower ¾ from the upper ¼ of the set is the third quartile.

Poorly correlated—If it is not possible to perform a “good” prediction, as defined via statistics, of one set of numbers from another set of numbers using a simple linear model, then the two sets of numbers are said to be poorly correlated.

Computer program—a written set of instructions that symbolically instructs an appropriately configured computer to execute an algorithm that will yield desired outputs from some set of inputs. The instructions may be written in one or several standard programming languages, such as C, C++, Visual BASIC, FORTRAN or the like. Alternatively, the instructions may be written by imposing a template onto a general-purpose numerical analysis program, such as a spreadsheet.

Experimental System Components

Small organic molecule—a compound of molecular weight less than 1500, preferably 100 to 1000, more preferably 300 to 600 such as biotin, fluorescein, rhodamine and other dyes, tetracycline and other protein binding molecules, and haptens, etc. The small organic molecule can provide a means for attachment of a nucleotide sequence to a label or to a support.

Support or surface—a porous or non-porous water insoluble material. The surface can have any one of a number of shapes, such as strip, plate, disk, rod, particle, including bead, and the like. The support can be hydrophilic or capable of being rendered hydrophilic and includes inorganic powders such as glass, silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. Natural or synthetic assemblies such as liposomes, phospholipid vesicles, and cells can also be employed. Binding of oligonucleotides to a support or surface may be accomplished by well-known techniques, commonly available in the literature. See, for example, A. C. Pease, et al, Proc. Nat. Acad. Sci. USA, 91:5022-5026 (1994).

Label—a member of a signal-producing system. Usually the label is part of a target nucleotide sequence or an oligonucleotide probe, either being conjugated thereto or otherwise bound thereto or associated therewith. The label is capable of being detected directly or indirectly. Labels include (i) reporter molecules that can be detected directly by virtue of generating a signal, (ii) specific binding pair members that may be detected indirectly by subsequent binding to a cognate that contains a reporter molecule, (iii) oligonucleotide primers that can provide a template for amplification or ligation or (iv) a specific polynucleotide sequence or recognition sequence that can act as a ligand such as for a repressor protein, wherein in the latter two instances the oligonucleotide primer or repressor protein will have, or be capable of having, a reporter molecule. In general, any reporter molecule that is detectable can be used. The reporter molecule can be isotopic or nonisotopic, usually non-isotopic, and can be a catalyst, such as an enzyme, a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, and the like. The reporter molecule can be a fluorescent group such as fluorescein, a chemiluminescent group such as luminol, a terbium chelator such as N-(hydroxyethyl)ethylenediaminetriacetic acid that is capable of detection by delayed fluorescence, and the like. The label is a member of a signal producing system and can generate a detectable signal either alone or together with other members of the signal producing system. As mentioned above, a reporter molecule can be bound directly to a nucleotide sequence or can become bound thereto by being bound to an sbp member complementary to an sbp member that is bound to a nucleotide sequence. Examples of particular labels or reporter molecules and their detection can be found in U.S. Pat. No. 5,508,178 issued Apr. 16, 1996, at column 11, line 66, to column 14, line 33, the relevant disclosure of which is incorporated herein by reference. When a reporter molecule is not conjugated to a nucleotide sequence, the reporter molecule may be bound to an sbp member complementary to an sbp member that is bound to or part of a nucleotide sequence.

Signal Producing System—the signal producing system may have one or more components, at least one component being the label. The signal producing system generates a signal that relates to the presence or amount of a target polynucleotide in a medium. The signal producing system includes all of the reagents required to produce a measurable signal. Other components of the signal producing system may be included in a developer solution and can include substrates, enhancers, activators, chemiluminescent compounds, cofactors, inhibitors, scavengers, metal ions, specific binding substances required for binding of signal generating substances, and the like. Other components of the signal producing system may be coenzymes, substances that react with enzymic products, other enzymes and catalysts, and the like. The signal producing system provides a signal detectable by external means, by use of electromagnetic radiation, desirably by visual examination. Signal-producing systems that may be employed in the present invention are those described more fully in U.S. Pat. No. 5,508,178, the relevant disclosure of which is incorporated herein by reference.

Ancillary Materials—Various ancillary materials will frequently be employed in the methods and assays utilizing oligonucleotide probes designed in accordance with the present invention. For example, buffers and salts will normally be present in an assay medium, as well as stabilizers for the assay medium and the assay components. Frequently, in addition to these additives, proteins may be included, such as albumins, organic solvents such as formamide, quaternary ammonium salts, polycations such as spermine, surfactants, particularly non-ionic surfactants, binding enhancers, e.g., polyalkylene glycols, or the like.

### DESCRIPTION OF EMBODIMENTS

In one embodiment the present invention provides a method of selecting a preferred set of oligomers from a large collection of oligomers such as a library of oligomers. A method involves choosing of a selection paradigm or selection algorithm that will be used as a predictor of oligo activity based on the selected target and properties and attributes of the oligo. The method of this embodiment further involves choosing another selection paradigm to apply against the group or set of oligos. A result of these two steps is two groups of selected oligos having predicted activity. The next step according to this embodiment of the invention is to apply a third selection paradigm, or algorithm against or to the combined grouping of the first two selected oligos providing thereby a third, most select group of oligos having predicted activity according to the chosen selection paradigms or algorithms. Moreover, the first selection paradigm, the second selection paradigm and the third selection paradigm may be the same or may be independently determined. The selection paradigms may be selected from the group consisting of decision tree, neural network, hierarchical clustering, clustering, regression tree, and combinations thereof.

An additional aspect of the present invention is directed to a method of selecting a predictive model from a master set or group of predictive models.

An additional embodiment of the present invention is directed to a database of oligomers and related indicia forming a decision tree predictive model. This database stores and correlates a plurality of attributes for a plurality of oligomers, which attributes consist of a flex-motif, an RNAse H motif, an amplicon, a feature, a sequence, an energy, a structure, an oligomer activity and a cell line. The database would further include an influence indicator, providing indication of the quantum of influence the attribute exerts on an oligomer activity. Moreover the database includes an activity manipulator for modulating the influence indicator where the activity manipulator modulates the influence indicator according to the influence of the oligomer attributes on the oligomer activity. These activity modulators may also be understood as a means of incorporating influence indicators in the dataset. These indicators provide additional information relative to the associated object or parameter and that objects quantum of influence on the specific attribute to which it is correlated.

In a yet further aspect of the present invention is directed to a computer system for selecting a set of oligomers having at least a threshold level of predicted activity according to one or more than one analytical paradigm, against a selected target.

In another aspect of the invention is described a system for designing a set of potentially active oligomers having at least a threshold level of predicted activity according to at least one design paradigms, against a target.

In yet another aspect of the present invention is described a method of selecting a set of active oligomers using a combination of more than one selection paradigms, through intersecting the results of selecting oligomer according to one or more selection algorithms and where the combination is synergistic.

In yet an additional aspect of the invention directed to a method of designing a potentially active oligomer for a target nucleic acid comprising determining a set of defining design attributes according to one or more than one design paradigms, a total nucleotide length for the potentially active oligomer and a threshold level of predicted activity for the potentially active oligomer. Combining a first and a second nucleotide according to the one or more than one design paradigms, thereby providing a first subset of the potentially active oligomer. Using an activity predicting system to determine the predicted activity of the first subset of the potentially active oligomer against the target and repeating these steps so long as the predictive activity remains at least equal to the threshold value and the number of combined nucleotides in the first subset is less then the total nucleotide length.

The present invention further provides methods of identifying a predictor of antisense oligonucleotide activity by identifying a plurality of properties for a plurality of oligonucleotides. The present invention further provides methods for selecting a predictive paradigm for an application of interest; evaluating oligonucleotide activity of a plurality of oligonucleotides, and correlating oligonucleotide activity for a plurality of oligonucleotides with the plurality of properties. A high correlation between oligonucleotide activity and a property indicates that the property is a predictor of antisense oligonucleotide activity.

The present invention provides methods of identifying predictors of antisense oligonucleotide activity. Upon selection of a biological target to which oligonucleotide binding is desired, a plurality of oligonucleotides are chosen, each of which is capable of hybridizing under physiological conditions to the biological target. Oligonucleotide target regions can be determined using feature-based or homology-based parameters.

Feature-based parameters include functional regions located on a particular biological target, such as, for example, the start codon, 3′ untranslated region 5′ untranslated region, poly A site, 3′ and 5′ splice sites, stop codon, boundries, coding region, introns, exons, intron-exon junctions and the like. Feature based parameters also include secondary structures such as stems, loops, hairpins, bulges and the like. Thus, feature-based parameters are those parameters that are based upon features of a particular biological target that are known and represent the traditional methodologies for selecting target regions for drug discovery.

Homology-based parameters are those parameters that are based upon particular regions of a particular biological target that are also present in additional species. Such regions are referred to as molecular interaction sites and are described in greater detail in, for example, U.S. Pat. No. 6,221,587, which is incorporated herein by reference in its entirety. Homology-based parameters are described below in greater detail. For a plurality of the oligonucleotides (i.e., two or more oligonucleotides) a plurality of properties is identified for each oligonucleotide. For example, where one hundred oligonucleotides are chosen for hybridization to a particular biological target, at least two properties are identified for each of at least two of the one hundred oligonucleotides. In some embodiments of the invention, a plurality of properties is identified for each oligonucleotide chosen to hybridize to a particular biological target. The number of oligonucleotides that are capable of hybridizing to a particular biological target, based upon nucleotide sequence alone, range from about 2 to about 10,000. When coupled with different nucleotide base and backbone chemistries, the number of oligonucleotides that are capable of hybridizing to a particular biological target increase dramatically.

Properties of oligonucleotides include, but are not limited to, hybridization position of oligonucleotide to its target, thermodynamics, number of nucleotide bases, proximity of binding to secondary structure of target, presence of oligonucleotide sequence motifs, pyridine content, A+T content, presence of RNAse cleavage sites, isoform specificity, cross-species activity, and oligonucleotide chemistry. In some embodiments, at least three, at least four, at least five, at least six, at least seven, at least eight, at least nine, at least ten, at least eleven, at least twelve, or all of the above-recited properties are identified for a plurality of oligonucleotides. One property of an oligonucleotide is its hybridization position with respect of its biological target. Such hybridization positions include, but are not limited to, the transcription start site, the 5′ cap site, the 5′ untranslated region, the start codon, the coding region, the stop codon, the 3′ untranslated region, 5′ splice sites, 3′ splice sites, specific exons, specific introns, mRNA stabilization signal sites, mRNA destabilization signal sites, poly-adenylation sites, and the gene sequence 5′ of known pre-mRNA. Any combination or all of these sites can be identified for any or all of the plurality of oligonucleotides. Such sites are often associated with a particular function. Another consideration is the position of the target site on the mRNA relative to functional sites such as the coding region. Antisense oligonucleotides that operate by an RNAse H mechanism seem to be affected little by target site function. Potent oligonucleotides have been reported for the coding regions, untranslated regions and even introns. On the other hand, antisense oligonucleotides that use a non-RNAse H mechanism are typically restricted to specific functional sites. Morpholino oligonucleotides, for example, inhibit via translation arrest and are often located near or upstream of the AUG initiation codon. Taylor et al., J. Biol. Chem., 1996, 271, 17445-52. They can also inhibit or alter splicing if placed at splice junctions. Schmajuk et al., Biol. Chem., 1.999, 274, 21783-9. Thus target site function becomes more important if a “steric blocking” mechanism of action is employed.

Another property of an oligonucleotide is its thermodynamic properties including, but not limited to, melting temperature (Tₘ,), association rates, dissociation rates, or any other physical property that can be predictive of oligonucleotide activity. The free energy of the biological target structure is defined as the free energy needed to disrupt any secondary structure in the target binding site of the biological target. This region includes any intra-target nucleotide base pairs that need to be disrupted for an oligonucleotide to bind to its complementary sequence. The effect of this localized disruption of secondary structure is to provide accessibility by the oligonucleotide. Such structures include, but are not limited to, double helices, terminal unpaired and mismatched nucleotides/loops, including hairpin loops, bulge loops, internal loops and multibranch loops, Serra et al., Methods in Enzymology, 1995, 259, 242.

The intermolecular free energies refer to inherent energy due to the most, stable structure formed by two oligonucleotides; such structures include dimer formation. Intermolecular free energies should also be taken into account when, for example, two or more oligonucleotides, of different sequence are to be administered to the same cell in an assay. The intramolecular free energies refer to the energy needed to disrupt the most stable secondary structure within a single oligonucleotide.

Such structures include, for example, hairpin loops, bulges and internal loops. The degree of intramolecular base pairing is indicative of the energy needed to disrupt such base pairing. The free energy of duplex formation is the free energy of denatured oligonucleotide binding to its denatured target sequence. The oligonucleotide-target binding is the total binding involved, and includes the energies involved in opening up intra- and inter-molecular oligonucleotide structures, opening up target structure, and duplex formation. The most stable RNA structure is predicted based on nearest neighbor analysis, Serra et al., Methods in Enzymology, 1995, 259, 242. This analysis is based on the assumption that stability of a given base pair is determined by the adjacent base pairs. For each possible nearest neighbor combination, thermodynamic properties have been determined and are provided. For double helical regions, two additional factors need to be considered, an entropy change required to initiate a helix and an entropy change associated with self-complementary strands only.

Thus, the free energy of a duplex can be calculated using the equation: ΔG°_{T}=ΔH°−TΔS°, where ΔG is the free energy of duplex formation, ΔH is the enthalpy change for each nearest neighbor, ΔS is the entropy change for each nearest neighbor, and T is temperature, The ΔH and ΔS for each possible nearest neighbor combination have been experimentally determined. These letter values are often available in published tables. For terminal unpaired and mismatched nucleotides, enthalpy and entropy measurements for each possible nucleotide combination are also available in published tables. Such results are added directly to values determined for duplex formation. For loops, while the available data is not as complete or accurate as for base pairing, one known model determines the free energy of loop formation as the sum of free energy based on loop size, the closing base pair, the interactions between the first mismatch of the loop with the closing base pair, and additional factors including being closed by AU or UA or a first mismatch of GA or UU. Such equations can also be used for oligoribonucleotide-target RNA interactions. The stability of DNA duplexes is used in the case of intra- or intermolecular oligodeoxyribonucleotide interactions. DNA duplex stability is calculated using similar equations as RNA stability, except experimentally determined values differ between nearest neighbors in DNA and RNA and helix initiation tends to be more favorable in DNA than in RNA. SantaLucia et al., Biochemistry, 1996, 35, 3555.

It has long been assumed that activity of an antisense oligonucleotide is directly related to the hybridization affinity of the oligonucleotide for its mRNA target. Support for this assumption comes from the observation that, at a given target site, longer oligonucleotides are more active than shorter ones. Baker et al., Biochimica et Biophysica Acta, 1999, 1489, 3-18. In addition, at a given site, oligonucleotide modifications that increase the melting temperature of the oligonucleotide-RNA duplex, often increase antisense activity and/or potency. Monia et al., J. Biol. Chem., 1993, 268, 14514-22; Altmann et al., Chimia, 1996, 50, 168-176; Wagner et al., Science, 1993, 260, 1510-3 and Schmajuk et al., J. Biot. Chem., 1999, 274, 21783-9. Mismatched oligonucleotides reduce the Tm and decrease the potency. Monia et al., J. Bio. Chem., 1992, 267, 19954-62; and Monia et al., Proc. Natl. Acad. Sci., 1996, 93, 1581-4 However, when comparing oligonucleotides targeted to different sites, Tm, alone is not sufficient to ensure activity. Chiang et al., J. Biol. Chem., 1.991, 266, 18162-71.

It has long been believed that secondary structure in the mRNA target affects hybridization affinity differently at different sites and thus affects antisense efficacy. Heikkila et al., Nature, 1.987, 328, 445-9; Jaroszewski et at., Antisense Res. Dev., 1993, 3, 339-48; Daaka et al., Oncogene Res., 1990, 5, 267-75; Rittner et at, Nuc. Acids Res., 1991, 19, 1421-6; and Sugimoto et al., 23rd Symposium on Nucleic Acids Chemistry, 1996, 175-76. Therefore methods for calculating RNA structure and calculating hybridization of the antisense oligonucleotide to the structured mRNA are useful for prediction of antisense activity. Early attempts by Stull et al. (Nuc. Acids Res., 1992, 20, 3501-8) found moderate correlation (R=0.66-0.99) between a predicted duplex score and antisense activity. Inclusion of an mRNA target secondary structure score in the calculation actually worsened correlation between calculated hybridization affinity and antisense activity. Since Stull's publication, improvements have been made to the rules and parameters for prediction of RNA secondary structure. Mathews et al., J. Mot. Biol., 1999, 288, 911-40. Effective parameters for prediction of DNA:RNA duplex stability are available (Sugimoto et al., Biochemistry, 1995, 34, 11211-6) and improved parameters for prediction of secondary structure in DNA oligonucleotides are also available; SantaLucia et al., Biochemistry, 1996, 35, 3555-62; Sugimoto et al., Nuc. Acids Res., 1996, 24, 4501-5; Allawi et al., Biochemistry, 1998, 37, 2170-9; Allawi et al., Nuc. Acids Res., 1998, 26, 2694-701; Allawi et al., Biochemistry, 1998, 37, 9435-44; and Peyret et al., Biochemistry, 1999, 38, 3468-77. Mathews et al. (RNA, 1999, 5, 1458-69) used these most up-to-date parameters to calculate equilibrium affinity of complementary DNA or RNA oligonucleotides to an RNA target talking into account the predicted stability of the oligonucleotide-target helix and the competition with predicted secondary structure of both the target and the oligonucleotide. When their predicted affinities were compared to antisense activity in one experiment (Ho et al. Nuc, Acids Res., 1996, 24, 1901-7), good correlation (R=0.91) was found between duplex free energy and antisense activity. when oligonucleotide self structure and/or target RNA structure were included in the calculation, antisense efficacy did not correlate with ΔG overall.

The reported correlations between predicted duplex stability and antisense activity may not always extend broadly to additional targets. When a data set of 349 antisense oligonucleotides targeting 12 genes (Giddings and Matveeva) was evaluated for correlation between duplex stability and antisense activity, the linear correlation coefficient was 0.22 suggesting that the strong correlations reported in earlier work may not always extend to larger data sets.

There are several possible explanations for the lack of a strong correlation between calculated hybridization of an oligonucleotide to its mRNA target and observed antisense activity. One possibility is that the calculated binding energies do not represent true equilibrium affinities. Although current algorithms are good enough to correctly predict 73% of base pairs in structures determined from comparative sequence analysis (J. Mol. Biol., 1999, 288, 911-40), this level of accuracy may not be enough to allow prediction of good antisense binding sites. In addition, current algorithms (Mathews et al., RNA, 1999, 5, 1458-69) use thermodynamic parameters for unmodified DNA or RNA when calculating free energies of antisense:RNA duplex formation or antisense oligonucleotide self structure.

Parameters determined from experiments using modified oligonucleotides could improve the predictions (Hashem et al., Biochemistry, 1998, 37, 61-72). Furthermore, parameters for predictions were measured in 1 M Na⁺, 0.1 mM EDTA and may not represent conditions of antisense binding. The large numbers of proteins involved in RNA synthesis, processing, transport, translation and degradation almost certainly affect binding of the antisense oligonucleotide to its target.

A second possibility is that the antisense target is pre-mRNA and secondary structures predicted for mRNAs are not representative of structures in pre-mRNAs. It is known that pre-RNA is the molecular target for many antisense oligonucleotides. Condon et al., J. Biol. Chem., 1996, 97 271, 30398-403 and Sierakowska et at., Methods Enzymol., 2000, 313, 506-21. The secondary structure of a pre-mRNA undergoing synthesis, processing and transport is likely not fully predictable from simple thermodynamic consideration.

The third, and most likely, possibility is that equilibrium affinity is not the sole factor impacting antisense activity. Tanaka et al., Nuc. Acids Symp. Ser., 1995, 34, 135-6. Oligonucleotide sequence and structure may affect properties of the antisense compound such as its affinity for proteins, ability to support RNAse H cleavage of the target, delivery to the cellular site of activity, and metabolic stability. These factors will, in turn, affect antisense activity. On the other hand, equilibrium affinity is not unimportant. When oligonucleotide sequence is kept constant, mRNA secondary structure affects antisense activity in a predictable way; activity is lower in structured targets than in unstructured ones. Vickers et al., Nuc. Acids Res., 2000, 28, 1340-1347.

Although factors other than target structure clearly play a role in antisense activity, predictions of local secondary structure have proven effective in identifying oligonucleotides with greater activity than those found by simple oligonucleotide “walks.” The strategy employed by Szakial and colleagues (Patzel et al., Nat. Biotechnol., 1998, 16, 64-8 and Patzel et al., Nuc. Acids Res., 1999, 27, 4328-34) searches for favorable local target elements, loops or bulges of about 10 nt, joints and terminal sequences. “Kissing” hairpins are known to be important for initiation of hybridization of long antisense RNAs (Tomizawa, Cell, 1986,47, 89-97 and Marino et al., Science, 1995, 268, 1448-54); these “favorable structures” may play a similar role for oligonucleotide hybridization. Additional thermodynamic parameters are used in the case of RNA/DNA hybrid duplexes. This would be the case for an RNA target and oligodeoxynucleotide. Such parameters were determined by Sugi moto et al. (Biochemistry, 1995, 34, 11211). In addition to values for nearest neighbors, differences were seen for values for enthalpy of helix initiation.

Another property of an oligonucleotide is its number of nucleotide bases. Oligonucleotides having few nucleotides (e.g., less than eight) may be non-selective and hybridize to a number of biomolecules. Alternately, oligonucleotides having many nucleotides (e.g., more than a few hundred) may not hybridize at all for a variety of reasons. Other lengths of oligonucleotides might be selected for non-antisense targeting strategies, for instance using the oligonucleotides as ribozymes. Such ribozymes normally require oligonucleotides of longer length as is known in the art.

Another property of an oligonucleotide is its proximity of binding to secondary structure of target. Exemplary secondary structures include, but are not limited to, bulges, loops, stems, pseudoknot, pseudo-halfknot, hairpins, knots, triple interacts, cloverleafs, or helices, or a combination thereof. Secondary structures are often critical to a particular function of an biological target. Thus, oligonucleotides that hybridize to locations proximal to such secondary structures may have greater activity.

Another property of an oligonucleotide is the presence of oligonucleotide sequence motifs. Sequence motifs include, for example, a string of four or three guanosine residues in a row, a string of adenosines, cytidines, uridines or thymidines, purines, pyrimidines, CG dl-nucleotide repeats, CA dinucleotide repeats, and UA or TA dinucleotide repeats. In addition, other sequence properties can be used as desired. These sequence motifs can be important in predicting oligonucleotide activity, or lack thereof. For example, U.S. Pat. No. 5,523,389 discloses oligonucleotides containing stretches of three or four guanosine residues in a row. Oligonucleotides having such sequences can act in a sequence-independent manner. For an antisense approach, such a mechanism is not usually desired. In addition, high numbers of dinucleotide repeats can be indicative of low complexity regions that can be present in large numbers of unrelated genes. It has been suggested that active oligonucleotides contain certain sequence motifs. Tu et al. (J. Biol. Chem., 1998, 273, 25125-31) report that TCCC is associated with antisense activity but no mechanism for this phenomenon was proposed. Smetsers et al. (Antisense Nucleic Acid Drug De v., 1996, 6, 63-7) previously reported that CCC is over-represented in the antisense oligonucleotides in their data set but that TCC is underrepresented. They suggest that over-represented motifs may be associated with protein-binding and non-antisense effects. Lesnik et al. (Biochemistry, 1995, 34, 10807-15) offered a very plausible explanation for the predominance of pyrimidines and especially C's in active oligonucleotides; that antisense activity is associated with high stability of the oligo:target hybrid relative to the alternative RNA:RNA duplex.

Motifs that support non-antisense effects exist. Non-antisense effects of G-rich 30 phosphorothioate oligonucleotides are well known (Ecker et al., Nuc. Acids Res., 1993, 21, 1853-6 and Bennett et al., Nuc. Acids Res., 1994, 22, 3202-9) and have been attributed to the tendency of these oligonucleotides to form G-quartet structures that then interfere with biological processes (Wyatt et al., In: Appl. Antisense Ther. Restenosis, 1990, 133-40). The simplest way to avoid these effects is to avoid G-rich oligonucleotides. Restricting oligonucleotides to less than 50% G with no strings and, at most, one G3 string usually does not detrimentally limit the number of oligonucleotides that can be selected from a target message. Homopolymers of other sequences also form unusual structures. Felsenfeld et al., Annu. Rev. Biochem., 1967, 36, 407-48. Although non-antisense effects of these structures are not well characterized, this should be considered when designing oligonucleotides rich in any single nucleotide or containing strings of any single structure.

Other motifs are also reported to produce non-antisense effects. Krieg et al, (Nature, 1995, 10 374, 546-9) reported that oligonucleotides containing CG, especially those with RRCGYY, can stimulate murine B cells in vitro and in vivo. The active motif in human cells is GTCGTT. Hartmann et al., J. Immunol., 2000, 164, 1617-24. To avoid designing any oligonucleotides containing the dinucleotide, CG, is, however, an overly stringent requirement. It eliminates nearly half the possible oligonucleotides that hybridize to a typical message from consideration, many of which show no immune stimulation at all. Therefore, it may be more prudent to avoid oligomers with the consensus hexamer motifs or to restrict the number of CG's in the sequence to less than two. In addition, the immunostimulatory effects of CG motifs are easily eliminated by chemical modification (e.g., 5-methyl C). Boggs et al., Antisense Nucleic Acid Drug Dev., 1997, 7, 461-71.

Another property of an oligonucleotide is pyrimidine content. Oligonucleotides with high pyrimidine content (70%-80%) are more likely to be active than oligonucleotides with lower pyrimidine content.

Another property of oligonucleotide is adenine and thymidine (A+T) content. Oligonucleotides with low A+T content (40%-50%) are more likely to be active than oligonucleotides with higher A+T content.

Another property of an oligonucleotide is presence of RNAse cleavage site. RNAse H is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Cleavage of the RNA target can be routinely detected by gel electrophoresis and, if necessary, associated nucleic acid hybridization techniques known in the art.

Another property of an oligonucleotide is isoform specificity. In the case of genes directing the synthesis of multiple transcripts, i.e. by alternative splicing, each distinct transcript is a unique target nucleic acid. If active compounds specific for a given transcript isoform are desired, the target nucleotide sequence can be limited to those sequences that are unique to that transcript isoform. If it is desired to modulate two or more transcript isoforms in concert, the target nucleotide sequence can be limited to sequences that are shared between the two or more transcripts. If sufficient sequence identity exists between two isoforms, it may be possible to identify an antisense oligonucleotide with activity against both targets. Using this strategy an oligonucleotide with good activity against both JNK-1 and JNK-2 was identified. Shan et al., Blood, 1999, 94, 4067-76. One attraction of antisense technology is that high specificity can be achieved. For example, inhibition of one isoform of a protein can be obtained without affecting another (Monia et al., Nat. Med., 1996, 2, 668-75; Bost et al., Mol. Cell. Biol., 1999, 19, 1938-49; and Dean et al., Proc Natl. Acad. Sci. 15 USA, 1994, 91, 11762-6). Such specificity is difficult to achieve with small molecule drugs. In order to obtain such specificity, one must be careful to design antisense oligonucleotides that will not hybridize to related mRNA sequences. Mitsuhashi, J. Gastroenterol., 1997, 32, 282-7. Since oligonucleotides with as few as three mismatches are reported to be inactive (Mania et al., Proc. Natl. Acad. Sci., 1996, 93, 1541-4), three mismatches to related targets should be sufficient but more would be desirable. Unfortunately, the most commonly used tool for identification of sequence homology, BLAST (Altschul et al., J. Mod. Biol., 1990, 215, 403-10), is ineffective at finding mismatched sites for oligonucleotides. A more effective technique for finding mismatched sites is to use BLAST to identify other mRNA sequences with homology to the target of interest and then to use a substring search to find mismatched sites in these mRNAs. Sites with zero or a few mismatches should be avoided.

Another property of an oligonucleotide is cross-species activity. Homology to analogous target sequences may also be desired. For example, an oligonucleotide can be selected to a region common to both humans and mice to facilitate testing of the oligonucleotide in both species. One feature of antisense inhibitors is that usually an active inhibitor of the human target is not an inhibitor of the same gene in mouse or another species. This is because mRNA sequences differ between species. It is sometimes possible, however, to select sites with high identity between two species and design oligonucleotides to those sites. If a sufficient number of such sites are tested it may be possible to identify an antisense oligonucleotide with activity in both species.

Another property of an oligonucleotide is its chemistry. Chemistries include, but are not limited to, oligonucleotides having modified internucleoside linkages, base modifications and sugar modifications. In the context of this invention, the term “oligonucleotide” is used to refer to an oligomer or polymer of ribonucleic acid (RNA) or deoxyribonucleic acid (DNA) or mimetics thereof. Thus, this term includes oligonucleotides composed of naturally-occurring nucleobases, sugars and covalent internucleoside (backbone) linkages as well as oligonucleotides having non-naturally-occurring portions that function similarly. Such modified or substituted oligonucleotides are often preferred over native forms, i.e., phosphodiester linked A, C, G, T and U nucleosides, because of desirable properties such as, for example, enhanced cellular uptake, enhanced affinity for nucleic acid target and increased stability in the presence of nucleases. A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a normal (where normal is defined as being found in RNA and DNA) pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage. Specific examples of oligonucleotide chemistries that can be defined as a property include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In addition to the base, sugar and internucleoside linkage, at each nucleoside position, one or more conjugate groups can be attached to the oligonucleotide via attachment to the nucleoside or attachment to the internucleoside linkage. For each nucleoside of an oligonucleotide, chemistry selection includes selection of the base forming the nucleoside from a large palette of different base units available. These may be “modified” or “natural” bases (also referenced herein as nucleobases) including the natural purine bases adenine and guanine, and the natural pyrimidine bases thymine, cytosine and uracil. They further can include modified nucleobases including other synthetic and natural nucleobases such as 5-methylcytosine (5-me-C), 5-hydroxymethyl cytosine, xanthine, hypoxanthine, 2-aminoadenine, methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracit), 4-thiouracil, 8-halo, 8-amino, 8-thiol, 8-thioalkyl, 8-hydroxyl and other 8-substituted adenines and guanines, 5-halo uracils and cytosines particularly 5-bromo, 5-trifluoromethyl and other 5-substituted uracils and cytosines, 7-methylguanine and 7-methyl adenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering, pages 858-859, Kroschwitz, U., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941 each of which is incorporated herein by reference. Oligonucleotide chemistry also includes selection of the sugar forming the nucleoside from a large palette of different sugar or sugar surrogate units available. These may be modified sugar groups, for instance sugars containing one or more substituent groups. Substituent groups comprise the following at the 2′ position: OH; F-; O-, S-, or N-alkyl, 0-, S-, or N-alkenyl, or 0, s- or N-alkynyl, wherein the alkyl, alkenyl and alkynyl may be substituted or unsubstituted C₂ to C₁₀ alkyl or C₂ to C₁₀ alkenyl and alkynyl. Also included are O((CH2)ₙO)ₘCH₃, O(CH₂)ₙOCH₃, O(CH2)ₙNH₂, O(CH2)ₙCH₃, O(CH₂)ₘONH₂, and O(CH₂)ₙON((CH₂)ₘCH₃))₂, where n and m are from 1 to about 10. Other substituent groups comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ON0₂, N0₂, N₃, NH₂, heterocycloalkyl, heterocycloalkaryl, aminoalkylamino, polyalkylamino, substituted silyl, an RNA cleaving group, a reporter group, an intercalator, and other substituents having similar properties. Another modification includes 2′methoxyethoxy (2′O—CH₂CH₂OCH₃), also known as 2′O-(2-methoxyethyl) or 2′MOE) (Martin et al., Hely. Chin. Acta, 1995, 78, 486) i.e., an alkoxyalkoxy group. A further modification includes 2′-dimethylaminooxyethoxy, i.e., a O(CH₂)₂ON(CH₃)₂ group, also known as 2′DMAOE. Other modifications include 2′-methoxy (2′-O—CH₃), 2′-aminopropoxy (2′-OCH₂CH₂CH₂NH₂) and 2′-fluoro (2′-F). Similar modifications can also be made at other positions on the sugar group, particularly the 3′ position of the sugar on the 3′ terminal nucleotide or in 2′-5′ linked oligonucleotides and the 5′ position of 5′ terminal nucleotide. The nucleosides of the oligonucleotides can also have sugar mimetics such as cyclobutyl moieties in place of the pentofuranosyl sugar Oligonucleotide chemistry also includes selection of the internucleoside linkage. These internucleoside linkages are also referred to as linkers, backbones or oligonucleotide backbones and include, but are not limited to, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoaiklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Internucleoside linkages for oligonucleotides that do not include a phosphorus atom therein, i.e., for oligonucleosides, have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, 0, S and CH₂ component parts. Oligonucleotide chemistry also includes oligonucleotide mimetics, in which the sugar and/or internucleotide linkage are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid PNA). In PNA compounds, the sugar-phosphate backbone of an oligonucleotide is replaced with an amide-containing backbone, in particular an aminoethylglycine backbone. The nucleobases are retained and are bound directly or indirectly to aza nitrogen atoms of the amide portion of the backbone.

Internucleoside linkages include, for example, oligonucleotides with phosphorothioate backbones and oligonucleosides with heteroatom backbones, and in particular —CH₂NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂ and —O—N(CH₃)—CH₂—C₁₋₁₂— (wherein the native phosphodiester backbone is represented as —O—P—O—CH₂—).

Oligonucleotide chemistry also includes attaching a conjugate group to one or more nucleosides or internucleoside linkages of an oligonucleotide. Modification of an oligonucleotide to chemically link one or more moieties or conjugates to the oligonucleotide can enhance the activity, cellular distribution or cellular uptake of the oligonucleotide. Such moieties include, bat are not limited to, lipid moieties such as a cholesterol moiety (Letsinger et al., Proc. Natl. Acad. Sci. USA, 1989, 86, 6553), cholic acid (Manoharan et al., Bioorg. Med. Chem. Let., 1994, 4, 1053), a thioether, e.g., hexyl-S-tritylthiol (Manoharan et al., Ann. N.Y. Acad., Sci., 1992, 660, 306; Manoharan et al., Bioorg. Med. Chem. Let, 1993, 3, 2765), a thiocholesterol (Oberhauser et al., Nuc. Acids Res., 1992, 20, 533), an aliphatic chain, e.g., dodecandiol or undecyl residues (Saison-Behmoaras er al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1, 2-di-O-hexadecyl-rac-glycero-3-H-phosphonate (Manoharan et al., Tetrahedron Lett., 1995, 36, 30 3651; Shea et al., Nuc. Acids Res., 199, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. 17 Biophys. Acta, 1.995, 1264, 229), or an octadecylamine or hexylamino-carbonyl-oxycholesterol moiety (Crooke et al., J. Pharmacol. Exp. Ther., 1996, 277, 923). For a particular oligonucleotide chemistry, it is not necessary for all positions in a given compound to be uniformly modified. In fact, more than one of the aforementioned modifications can be incorporated in a single compound or even at a single nucleoside within an oligonucleotide. Oligonucleotide chemistry also includes compounds that are chimeric compounds, “Chimeric” compounds or “chimeras,” in the context of this invention, are compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid, An additional region of the oligonucleotide can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. Chimeric oligonucleotides include composite structures representing the union of two or more oligonucleotides, modified oligonucleotides, oligonucleosides and/or oligonucleotide mimetics as described above. Such compounds have also been referred to in the art as “hybrids” or “gapmers”. Representative United States patents that teach the preparation of such hybrid structures include, but are not limited to, U.S. Pat. Nos. 5,013,830; 5,149,797, 5,220,007; 5,256,775; 5,366,878; 5,403,711; 5,491,133; 5,565,350; 5,623,065; 5,652,355; 5,652,356; and 5,700,922, each of which is incorporated herein by reference. Other properties of oligonucleotides include those properties that have not yet been assigned but which are suspected to be a property. For example, there may be some feature or characteristic of an oligonucleotide that has not yet been associated with oligonucleotide activity. These properties can be identified as predictors for oligonucleotide activity using the methods described herein. Upon identification of a plurality of properties, a plurality of oligonucleotides is evaluated for oligonucleotide activity. At least two oligonucleotides are evaluated for activity. In some embodiments of the invention, at least fifty percent, at least sixty percent at least seventy percent, at least eighty percent, at least ninety percent, or all oligonucleotides are evaluated for oligonucleotide activity.

Oligonucleotide activities include, but are not limited to modulation of protein synthesis, modulation of mRNA modulation of cell viability, modulation of microRNA, miRNA, combinations thereof and the modulation of related nucleic acids.

Oligonucleotide-mediated modulation of expression of a target nucleic acid can be assayed in a variety of ways known in the art. For example, target RNA levels can be quantitated by Northern blot analysis, competitive PCR, or reverse transcriptase polymerase chain reaction (RTPCR). RNA analysis can be performed on total cellular RNA or, in the case of polypeptide-encoding nucleic acids, poly(A)+mRNA. Reverse transcriptase polymerase chain reaction (RT-PCR) can be conveniently accomplished using the commercially available ABI PRISM 7700 Sequence Detection System (PE-Applied Biosystems, Foster City, Calif.) according to manufacturer's instructions. Other methods of PCR are also known in the art. Target protein levels can be quantitated in a variety of ways well known in the art, such as immunoprecipitation, Western blot analysis (immunoblotting), Enzyme-linked immunosorbent assay (ELISA) or fluorescence-activated cell sorting (FRCS). Antibodies directed to a protein encoded by a target nucleic acid can be identified and obtained from a variety of sources, such as the MSRS catalog of antibodies, (Aerie Corporation, Birmingham, Mich.), or can be prepared via conventional antibody generation methods. Methods for preparation of polyclonal, monospecific and monoclonal antisera are taught by, for example, Ausubel et al. (Short Protocols in Molecular Biology, 2nd Ed., pp 11-3 to 11-54, Greene Publishing Associates and John Wiley & Sons, New York, 1992). Immunoprecipitation methods are standard in the art and are described by, for example, Ausubel et al. (Id., pp. 10-57 to 1043). Western blot (immunoblot) analysis is standard in the art 30 (Id., pp. 132 to 10-10-35). Enzyme-linked immunosorbent assays (ELISA) are standard in the art (Id., pp. 11-5 to II-17). Once a plurality of properties for a plurality of oligonucleotides have been identified and the oligonucleotide activity for a plurality of oligonucleotides has been evaluated, oligonucleotide activity for a plurality of oligonucleotides is correlated with the plurality of properties. A high correlation between oligonucleotide activity and a property indicates that the property is a predictor of antisense oligonucleotide activity. Correlation can be accomplished by, for example, creating a hierarchy of oligonucleotide activity. Oligonucleotides can be ranked in the hierarchy according to the extent of oligonucleotide activity. Each oligonucleotide is associated with a plurality of properties, as described above Those properties associated with oligonucleotides at the top of the hierarchy (i.e., those with the highest activity) are predictors of oligonucleotide activity. One skilled in the art can set a minimum activity below which the associated properties are not considered to be predictors of oligonucleotide activity. For example, properties primarily associated with oligonucleotides within the bottom 25% may be excluded from being predictors. In addition, the percentage of a particular property within a particular segment of the hierarchy can be an indicator of the strength of the predictor. For example, 75% of particular property associated with the top 15% of the hierarchy would indicate that the particular property is a better predictor of oligonucleotide activity than a second property, wherein 45% of the second property is associated with the top 15% of the hierarchy. In some embodiments of the invention, the hierarchy can be optimized to allow complex combinations of the properties to be analyzed. Thus, combinations of at least two different properties can be analyzed for their ability as a combination to act as predictors for oligonucleotide activity. In addition, synergy among a plurality of properties can be identified in this manner. Optimization can be achieved by, for examples evolutionary programming, neural nets, and the like.

In some embodiments of the invention, a new property is identified that is correlated with oligonucleotide activity. The methods of the invention can be practiced using the new property. The present invention also provides methods of enhancing identification of an active oligonucleotide by eliminating the oligonucleotides in the hierarchy that have little or no activity. For example, elimination of oligonucleotides in the bottom five percent of the hierarchy enhances identification of an active oligonucleotide. Likewise, the present invention also provides methods of enhancing identification of an active oligonucleotide by selecting oligonucleotides which have much activity. For example, selecting at least one oligonucleotide from the top five percent of oligonucleotides in the hierarchy enhances identification of an active oligonucleotide. Enhancement of oligonucleotides with activity enhances the ability to identify predictors of oligonucleotide activity.

The biological target, or regions thereof, can be determined by homology-based parameters. Briefly, the nucleotide sequence of the target nucleic acid is compared with the nucleotide sequences of a plurality of nucleic acids from different taxonomic species. The target nucleic acid can be present in eukaryotic cells or prokaryotic cells, the target nucleic acid can be bacterial or viral as well as belonging to a “higher” organism such as human.

Any type of nucleic acid can serve as a target nucleic acid, including, but are not limited to, messenger RNA (mRNA), pre-messenger RNA (pre-mRNA), transfer RNA (NA), ribosomal RNA (rRNA), microRNA (miRNA) or small nuclear RNA (snRNA). Initial selection of a particular target nucleic acid can be based upon any functional criteria. Nucleic acids known to be important during inflammation, cardiovascular disease, pain, cancer, arthritis, trauma, obesity, Huntingtons, neurological disorders, or other diseases or disorders, for example, are exemplary target nucleic acids. Nucleic acids known to be involved in pathogenic genomes such as, for example, bacterial, viral and yeast genomes are exemplary prokaryotic nucleic acid targets. Pathogenic bacteria, viruses and yeast are well known to those skilled in the art.

Additional nucleic acid targets can be determined independently or can be selected from publicly available prokaryotic and eukaryotic genetic databases known to those skilled in the art. Preferred databases include, for example, Online Mendelian Inheritance in Man (OMIM), the Cancer Genome Anatomy Project (CLAP), GenBank, EMBL, PIR, SWISS-PROT, and the like. In addition, nucleic acid targets can also be selected from private genetic databases. Alternatively, nucleic acid targets can be selected from available publications or can be determined especially for use in connection with the present invention.

After a nucleic acid target is selected or provided, the nucleotide sequence of the nucleic acid target is determined and then compared to the nucleotide sequences of a plurality of nucleic acids from different taxonomic species. The nucleotide sequence of the nucleic acid target can be determined by scanning at least one genetic database or is identified in available publications. Databases known and available to those skilled in the art include, for example, the Expressed Gene Anatomy Database (EGAD) and Unigene-Homo Sapiens database (Unigene), GenBank, and the like. These databases can be used in connection with searching programs such as, for example, Entrez, which is known and available to those skilled in the art, and the like. Preferably, the most complete nucleic acid sequence representation available from various databases is used. Alternatively, partial nucleotide sequences of nucleic acid targets can be used when a complete nucleotide sequence is not available. The nucleotide sequence of the nucleic acid target can also be determined by assembling a plurality of overlapping expressed sequence tags (ESTs).

The EST database (dbEST), which is known and available to those skilled in the art, comprises approximately one million different human mRNA sequences comprising from about 500 to 1000 nucleotides, and various numbers of ESTs from a number of different organisms. Assembly of overlapping ESTs extended along both the 5′ and 3′ directions results in a full-length “virtual transcript.” The resultant virtual transcript can represent an already characterized nucleic acid or can be a novel nucleic acid with no known biological function. The Institute for Genomic Research Human Genome Index (HGI) database, which is known and available to those skilled in the art, contains a list of human transcripts. The nucleotide sequence of the nucleic acid target is compared to the nucleotide sequences of a plurality of nucleic acids from different taxonomic species. A plurality of nucleic acids from different taxonomic species, and the nucleotide sequences thereof, can be found in genetic databases, from available publications, or can be determined especially for use in connection with the present invention. The nucleic acid target can be compared to the nucleotide sequences of a plurality of nucleic acids from different taxonomic species by performing a sequence similarity search, an ortholog search, or both, such searches being known to persons of ordinary skill in the art. The result of a sequence similarity search is a plurality of nucleic acids having at least a portion of their nucleotide sequences which are homologous to at least an 8 to 20 nucleotide region of the target nucleic acid, referred to as the window region. Preferably, the plurality of nucleotide sequences comprise at least one portion which is at least 60%, at least 70%, at least 80%, or at least 90% homologous to any window region of the target nucleic acid. Sequence similarity searches can be performed manually or by using several available computer programs known to those skilled in the art. Preferably, Blast and Smith-Waterman algorithms, which are available and known to those skilled in the art, and the like can be used. The GCG Package provides a local version of Blast that can be used either with public domain databases or with any locally available searchable database_{—}22 GCG Package v. 9.0 is a commercially available software package that contains over 100 interrelated software programs that enables analysis of sequences by editing, mapping, comparing and aligning them. Other programs included in the GCG Package include, for example, programs that facilitate RNA secondary structure predictions, nucleic acid fragment assembly, and evolutionary analysis. Another alternative sequence similarity search can be performed, for example, by BlastParse.

BlastParse is a PERL script running on a UNIX platform that automates the strategy described above. BlastParse parses all the GenBank fields into tab-delimited text that can then be saved in a relational database format for easier search and analysis, which provides flexibility. The end result is a series of completely parsed GenBank records that can be easily sorted, filtered, and queried against, as well as an annotations-relational database.

Another toolkit capable of doing sequence similarity searching and data manipulation is SEATS, also from NCBI. This tool set is written in PERL and C and can run on any computer platform that supports these languages. This toolkit provides access to Blast2 or gapped Blast. The plurality of nucleic acids from different taxonomic species that have homology to the target nucleic acid, as described above in the sequence similarity search, can be further delineated so as to find orthologs of the target nucleic acid therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing an ortholog search, an exhaustive list of homologous sequences from diverse organisms is obtained. Subsequently, these sequences are analyzed to select the best representative sequence that fits the criteria for being an ortholog.

An ortholog search can be performed by programs available to those skilled in the art including, for example, Compare. Preferably, an ortholog search is performed with access to complete and parsed GenBank annotations for each of the sequences. Currently, the records obtained from GenBank are “flat-files,” and are not ideally suited for automated analysis. The ortholog search can be performed using a Q-Compare program. The above-described similarity searches provide results based on cut-off values, referred to as e-scores. E-scores represent the probability of a random sequence match within a given window of nucleotides. The lower the e-score, the better the match. One skilled in the art is familiar with e-scores. The user defines the e-value cut-off depending upon the stringency, or degree of homology desired, as described above. In embodiments of the invention where prokaryotic molecular interaction sites are identified, it is preferred that any homologous nucleotide sequences that are identified be non-human. The sequences required can be obtained by searching ortholog databases. One such database is Hovergen, which is a curated database of vertebrate orthologs. Ortholog sets can be exported from this database and used as is, or used as seeds for further sequence similarity searches as described above. Further searches can be desired, for example, to find invertebrate orthologs A database of prokaryotic orthologs, COGS, is available and can be used interactively on the internet. The nucleotide sequences of a plurality of nucleic acids from different taxonomic species can be compared to the nucleotide sequence of the target nucleic acid by performing a sequence similarity search using dbEST, or the like, and constructing virtual transcripts. Using EST information is useful for two distinct reasons. First, the ability to identify orthologs for human genes in evolutionarily distinct organisms in GenBank database is limited. As more effort is directed towards identifying ESTs from these evolutionarily distinct organisms, dbEST is likely to be a better source of ortholog information. A sequence similarity search can be performed using Smith-Waterman algorithms, as described above, under high stringency against dbEST excluding human sequences. A full-length or partial “virtual transcript” for non-human RNAs is constructed by a process whereby overlapping EST sequences are extended along both the 5′ and 3′ directions, until a “full-length” transcript is obtained. A chimeric virtual transcript can also be constructed. The resultant virtual transcript can represent an already characterized RNA molecule or could be a novel RNA molecule with no known biological function. TIGR HGI database makes available an engine to build virtual transcripts called TIGR-Assembler. GLAXO-MRC and GeneWorld from Pangea provide for construction of virtual transcripts as well. Find Neighbors and Assemble EST Blast can also be used to build virtual transcripts. After the orthologs or virtual transcripts described above are obtained through either the sequence similarity search or the ortholog search, at least one sequence region that is conserved among the plurality of nucleic acids from different taxonomic species and the target nucleic acid is identified. Interspecies sequence comparisons can be performed using numerous computer programs which are available and known to those skilled in the art. Interspecies sequence comparison can be performed using Compare, which is available and known to those skilled in the art. Compare is a GCG tool that allows pair-wise comparisons of sequences using a window/stringency criterion. Compare produces an output file containing points where matches of specified quality are found. These can be plotted with another GCG tool, DotPlot. Alternatively, the identification of a conserved sequence region can be performed by interspecies sequence comparisons using the ortholog sequences generated from Q-Compare in combination with CompareOverWins. Preferably, the list of sequences to compare, i.e., the ortholog sequences, generated from Q-Compare can be entered into the CompareOverWins algorithm. interspecies sequence comparisons can be performed by a pair-wise sequence comparison in which a query sequence is slid over a window on the master target sequence. The window can be from about 9 to about 99 contiguous nucleotides. Sequence homology between the window sequence of the target nucleic acid and the query sequence of any of the plurality of nucleic acid sequences obtained as described above, can be at least 60%, at least 70%, at least 80%, and at least 90%. The most preferable method of choosing the threshold is to have the computer automatically try all thresholds from 50% to 100% and choose a threshold based on a metric provided by the user. One such metric is to pick the threshold such that exactly n hits are returned, where n is usually set to 3. This process is repeated until every base on the query nucleic acid, which is a member of the plurality of nucleic acids described above, has been compared to every base on the master target sequence. The resulting scoring matrix can be plotted as a scatter plot. Based on the match density at a given location, there may be no dots, isolated dots, or a set of dots so close together that they appear as a line. The presence of lines, however small, indicates primary sequence homology. Sequence conservation within nucleic acid molecules, particularly the UTRs of RNA, in divergent species is likely to be an indicator of conserved regulatory elements that are also likely to have a secondary structure. The results of the interspecies sequence comparison can be analyzed using MS Excel and visual basic tools in an entirely automated manner as known to those skilled in the art. After at least one region that is conserved between the nucleotide sequence of the nucleic acid target and the plurality of nucleic acids from different taxonomic species, preferably via the orthologs, is identified, the conserved region is analyzed to determine whether it contains secondary structure. Determining whether the identified conserved regions contain secondary structure can be performed by a number of procedures known to those skilled in the art. Determination of secondary structure is preferably performed by self complementarity comparison, alignment and covariance analysis, secondary structure prediction, or a combination thereof.

Secondary structure analysis can be performed by alignment and covariance analysis. Numerous protocols for alignment and covariance analysis are known to those skilled in the art. Preferably, alignment is performed by ClustalW, which is available and known to those skilled in the art. ClustalW is a tool for multiple sequence alignment that, although not a part of GCG, can be added as an extension of the existing GCG tool set and used with local sequences. ClustalW is described in Thompson et al., Nuc. Acids Res., 1994, 22, 4673-4680, which is incorporated herein by reference in its entirety. These processes can be scripted to automatically use conserved UTR regions identified in earlier steps. Seqed, a UNIX command line interface available and known to those skilled in the art, allows extraction of selected local regions from a larger sequence. Multiple sequences from many different species can be clustered and aligned for further analysis. The output of all possible pair-wise CompareOverWindows comparisons can be compiled and aligned to a reference sequence using a program called AlignHits. One purpose of this program is to map all hits made in pair-wise comparisons back to the position on a reference sequence. This method combining CompareOverWindows and AlignHits provides more local alignments (over 20-100 bases) than any other algorithm. This local alignment is required for the structure finding routines described later such as covariation or RevComp. This algorithm writes a Fasta file of aligned sequences. The algorithm does not correct single base insertions or deletions. This is usually accomplished by putting the output through ClustalW described elsewhere. It is important to differentiate this from using ClustalW by itself, without CompareOverWindows and AlignHits. Covariation is a process of using phylogenetic analysis of primary sequence information for consensus secondary structure prediction. Covariation is described in the following references, each of which is incorporated herein by reference in their entirety: Gutell et al., “Comparative Sequence Analysis Of Experiments Performed During Evolution” In Ribosomal RNA Group I Introns, Green, Ed., Austin:Landes, 1996; Gautheret et al., Nuc. Acids Res., 1997, 25, 1559-1564; Gautheret et al., RNA, 1995, 1, 807-814; Lodmell et al., Proc. Nat!. Acad. Sci. USA, 1995, 92, 10555.10559; Gautheret et al., J. Mol. Biol., 1995, 248, 27.43; Gutell, Nuc. Acids Res., 1994, 22, 3502-3517; Gutell, Nuc. Acids Res., 1993, 21, 3055-3074; Gutell, Nuc. Acids Res., 1993, 21, 3051-3054; Woese, Proc. Natd. Acad. Sci. USA, 1989, 86, 3119-3122; and Woese et al., Nuc Acids Res., 1980, 8, 2275-2293, each of which is incorporated herein by reference in its entirety. Covariance software can be used for covariance analysis. Covariation, a set of programs for the comparative analysis of RNA structure from sequence alignments, can be used. Covariation uses phylogenetic analysis of primary sequence information for consensus secondary structure prediction. A complete description of a version of the program has been published (Brown, J. W., Phylogenetic analysis of RNA structure on the Macintosh computer, CABIOS, 1991, 7, 391-393). The current version is v4.1, which can perform various types of covariation analysis from RNA sequence alignments, including standard covariation analysis, the identification of compensatory base-changes, and mutual information analysis. The program is well-documented and comes with extensive example files. It is compiled as a stand-alone program; it does not require Hypercard (although a much smaller “stack” version is included). This program will run in any Macintosh environment running MacOS 5 v7.1 or higher. Faster processor machines (68040 or PowerPC) is suggested for mutual information analysis or the analysis of large sequence alignments. Secondary structure analysis can be performed by secondary structure prediction. There are a number of algorithms that predict RNA secondary structures based on thermodynamic parameters and energy calculations. Secondary structure prediction can be performed using either M-fold or RNA Structure 2. 52 M-fold is available as a part of GCG package. RNA Structure 2. 52 is a windows adaptation of the M-fold algorithm. Secondary structure analysis can also be performed by self complementarity comparison. Self complementarily comparison can be performed using Compare, described above. Compare can be modified to expand the pairing matrix to account for G-U or UG basepairs in addition to the conventional Watson-Crick G-C/C-G or A-U/U-A pairs. Such a modified Compare program (modified Compare) begins by predicting all possible base-pairings within a given sequence. As described above, a small but conserved region, preferably a UTR, is identified based on primary sequence comparison of a series of orthologs. In modified Compare, each of these sequences is compared to its own reverse complement. Allowable base-pairings include Watson-Crick A-U, G-C pairing and non-canonical G-U pairing. An overlay of such self complementarity plots of all available orthologs, and selection for the most repetitive pattern in each, results in a minimal number of possible folded configurations. These overlays can then be used in conjunction with additional constraints, including those imposed by energy considerations described above, to deduce the most likely secondary structure. The output of AlignHits is read by a program called RevComp. A preferred purpose of this program is to use base pairing rules and ortholog evolution to predict RNA secondary structure. RNA secondary structures are composed of single stranded regions and base paired regions, called stems. Since structure conserved by evolution is searched, the most probable stem for a given alignment of ortholog sequences is the one that could be formed by the most sequences Possible stem formation or base pairing rules is determined by, for example, analyzing base pairing statistics of stems which have been determined by other techniques such as NMR. The output of RevComp is a sorted list of possible structures, ranked by the percentage of ortholog set member sequences that could form this structure. Because this approach uses a percentage threshold approach, it is insensitive to noise sequences. Noise sequences are those that either not true orthologs, or sequences that made it into the output of AlignHits due to high sequence homology even though they do not represent an example of the structure that is searched.

A very similar algorithm is implemented using Visual basic for Applications (VBA) and Microsoft Excel to be run on PCs, to generate the reverse complement matrix view for the given set of sequences. A result of the secondary structure analysis described above, whether performed by alignment and covariance, self complementarity analysis, secondary structure predictions, such as using M-fold or otherwise, is the identification of secondary structure in the conserved regions among the target nucleic acid and the plurality of nucleic acids from different taxonomic species. Exemplary secondary structures that may be identified include, but are not limited to, bulges, loops, stems, hairpins, knots, triple interacts, cloverleafs, or helices, or a combination thereof. Alternatively, new secondary structures may be identified. Once the secondary structure of the conserved region has been identified, as described above, at least one structural motif for the conserved region having secondary structure can be identified. These structural motifs correspond to the identified secondary structures described above. For example, analysis of secondary structure by self complementation may provide one type of secondary structure, whereas analysis by M-fold may provide another secondary structure. All the possible secondary structures identified by secondary structure analysis described above can, thus, be represented by a family of structural motifs. Once the secondary structure(s) of the target nucleic acids, as well as the secondary structures of nucleic acids from different taxonomic species, have been identified, further nucleic acids can be identified by searching on the basis of structure, rather than by primary nucleotide sequence, as described above. Additional nucleic acids which have secondary structure similar or identical to the secondary structure found as described above can be identified by constructing a family of descriptor elements for the structural motifs described above, and identifying other nucleic acids having secondary structures corresponding to the descriptor elements.

The combination of any or all of the nucleic acids having secondary structure can be compiled into a database. The entire process can be repeated with a different target nucleic acid to generate a plurality of different secondary structure groups that can be compiled into the database. Thus, databases of molecular interaction sites can be compiled by performing by the invention described herein. After the hypothetical structure motifs are determined from the secondary structure analysis described above, a family of structure descriptor elements can be constructed. The structural motifs described above can be converted into a family of descriptor elements. One skilled in the art is familiar with construction of descriptors. Structure descriptors are described in, for example, Laferriere et at., Comput Appl. Biosci., 1994, 10, 211-212, incorporated herein by reference in its entirety. A different structure descriptor element is constructed for each of the structural motifs identified from the secondary structure analysis.

Briefly, the secondary structure is converted to a generic text string. For novel motifs, further biochemical analysis such as chemical mapping or mutagenesis may be needed to confirm structure predictions. Descriptor elements may be defined to have various stringency. In addition, the descriptor elements can be defined to allow for a wobble. Thus, descriptor elements can be defined to have any level of stringency desired by the user. After a family of structure descriptor elements is constructed, nucleic acids having secondary structure which correspond to the structure descriptor elements can be identified. Nucleic acids having secondary structure that correspond to the structure descriptor elements are identified by searching at least one database, performing clustering and analysis, identifying orthologs, or a combination thereof. Thus, the identified nucleic acids have secondary structure that falls within the scope of the secondary structure defined by the descriptor elements. Thus, the identified nucleic acids have secondary structure identical to nearly identical, depending on the stringency of the descriptor elements, to the target nucleic acid. Nucleic acids having secondary structure that correspond to the structure descriptor elements can be identified by searching at least one database. Any genetic database can be searched. Preferably, the database is a UTR database, which is a compilation of the untranslated regions in messenger RNAs.

Preferably the database is searched using a computer program, such as, for example, Rnamot, a UNIX-based motif searching tool available from Daniel Gautheret. Each “new” sequence that has the same motif is then queried against public domain databases to identify additional sequences. Results are analyzed for recurrence of pattern in UTRs of these additional ortholog sequences, as described below, and a database of RNA secondary structures is built. One skilled in the art is familiar with Rnamot. Briefly, Rnamot takes a descriptor string and searches any Fasta format database for possible matches. Descriptors can be very specific, to match exact nucleotide(s), or can have built-in degeneracy. Lengths of the stem and loop can also be specified. Single stranded loop regions can have a variable length. G-U pairings are allowed and can be specified as a wobble parameter. Allowable mismatches can also be included in the descriptor definition. Functional significance is assigned to the motifs if their biological role is known based on previous analysis. Nucleic acids identified by searching databases such as, for example, searching a UTR database using Rnamot, can be clustered and analyzed so as to determine their location within the genome. The results provided by Rnamot simply identify sequences containing the secondary structure but do not give any indication as to the location of the sequence in the genome. Clustering and analysis is preferably performed with ClustalW, as described above. After clustering and analysis is performed as described above, orthologs can be identified as described above. However, in contrast to the orthologs identified above, which were solely identified on the basis of their primary nucleotide sequences, these new orthologous sequences are identified on the basis of structure using the nucleic acids identified using Rnamot. Identification of orthologs is preferably performed by BlastParse or Q-Compare, as described above. Once the biological target has been selected, oligonucleotides directed to the target regions are prepared. The oligonucleotides can be prepared by standard, automated means. The oligonucleotides can be synthesized as a particular group or as a combinatorial library. The oligonucleotides can be synthesized on various automated synthesizers. For illustrative purposes, the synthesizer utilized for synthesis of above described libraries, is a variation of the synthesizer described in U.S. Pat. Nos. 5,472,672 and 5,529,756, the entire contents of which are herein incorporated by reference. The synthesizer described in those patents was modified to include movement in along the Y axis in addition to movement along the X axis. As so modified, a 96-well array of compounds can be synthesized by the synthesizer. The synthesizer can further include temperature control and the ability to maintain an inert atmosphere during all phases of a synthesis. The reagent array delivery format employs orthogonal X-axis motion of a matrix of reaction vessels and Y-axis motion of an array of reagents. Each reagent has its own dedicated plumbing system to eliminate the possibility of cross-contamination of reagents and line flushing and/or pipette washing. This in combined with a high delivery speed obtained with a reagent mapping system allows for the extremely rapid delivery of reagents. This further allows long and complex reaction sequences to be performed in an efficient and facile manner. Such procedures are described in more detail in, for example, U.S. patent application Ser. No. 09/076,404, which is incorporated herein by reference in its entirety.

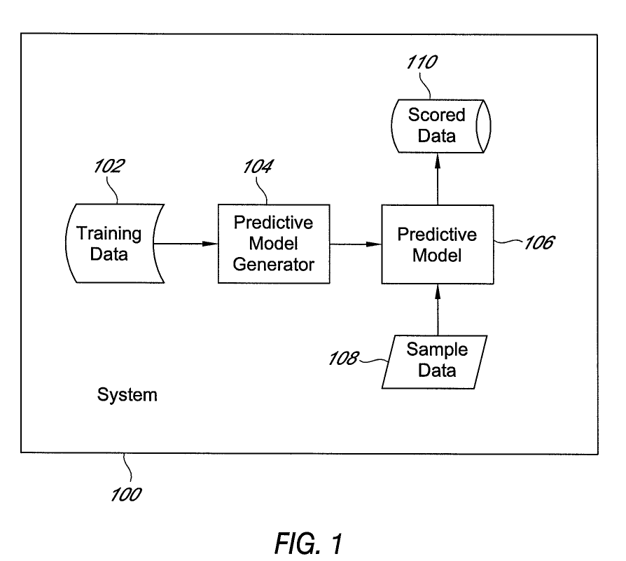

FIG. 1 illustrates a block diagram of a system 100 in accordance with an embodiment of the present invention. A predictive model generator 104 uses training data 102 to generate a predictive model 106. Predictive model 106 receives oligonucleotide sample data 108 and scores it. The scored data is reflective of a likelihood that the oligonucleotide will show activity against a specified target. In the illustrated embodiment, scored data is out put to a data store 110, although in alternative embodiments the scored data can be presented in another fashion, for example by output to a display screen.

While preferred embodiments of the invention have been described using antisense as a model, one of ordinary skill readily will appreciate that the methods, algorithms, and teachings of the specification readily are applicable to identification and optimization of oligonucleotides having other activities such as, e.g., RNAi properties, ribozyme properties as well as other catalytic, structural or modulatory properties that can be created using oligonucleotides or oligonucleotide-like molecules such as, e.g., peptide nucleic acids.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

The following examples are directed to the selection of one or more data mining methods from those available in the art. Although the selection of a predictive algorithm must be selected in view of the context and is a difficult one, according to methods of the present invention and according to the following examples, a predictive algorithm suitable for the desired task may be obtained.

Furthermore it is envisioned according to the present invention that during the practice of several embodiments of the present invention that additional relationships and properties will be determined to be significant or to have substantial correlation to activity. The active oligomers provided through any analysis, such as statistical, of the oligomers as part of the database will provide or reveal additional parameters that may only have activity for a specific target. Importantly, the determination of new parameters as derived by from database correlations as revealed through practice of the methods of the present invention are envisioned and provided as part of the methods of the present invention.

### Example 1

After testing a variety of data mining methods, the decision learning induction method to predict oligomer activity was selected for study. As is known by those of skill in the art, decision trees are typically used for inductive inference and can approximate discrete value functions. In comparison to neural networks, regression trees and other methods, the decision tree method is very successful at learning patterns in data in the given dataset, as well as presenting the output in a readable form. The output model of a decision tree learning method is a tree having a hierarchy of attributes, each of which splits the data in the best way at that point in time (the tree is built from the root down), and the leaves that classify the oligomer instances.

After initial cleaning and filtering of a part of the Isis Pharmaceuticals proprietary screening data, the data was classified into two categories: Active and Inactive, and was ready to train. In the training and learning phase, we tested a variety of configurations and parameters set options, which concluded in creation of out best performing model.

We present the resulting model created using the decision tree learning method, and evaluated with 10-fold stratified cross-validation. Cur model evaluated to 66% of correctly classified instances, tested using 10-fold cross-validation. Compared to state-of-the-art model in the literature (Giddings et al, NAR 2002) that evaluated at 53% cross-validation, we obtained an increase of 25% in the performance.

TABLE 1.1
Detailed Accuracy by Class

| TP Rate | FP Rate | Precision | Recall | F-Measure | Class |
|---|---|---|---|---|---|
| 64.5% | 33% | 60.3% | 64.5% | 62.4% | Active |
| 67% | 35.5% | 70.9% | 67% | 68.9% | Inactive |

TABLE 1.2
Confusion Matrix

| Active | Inactive | ← | classified as |
|---|---|---|---|
| 1619 | 890 | \| | Active |
| 1065 | 2167 | \| | Inactive |

TABLE 1.3
Predictive Model of Antisense Oligomer Activity
(attribute values normalized to [0,1])

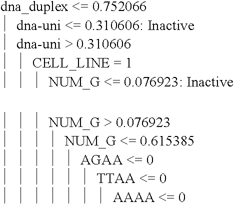

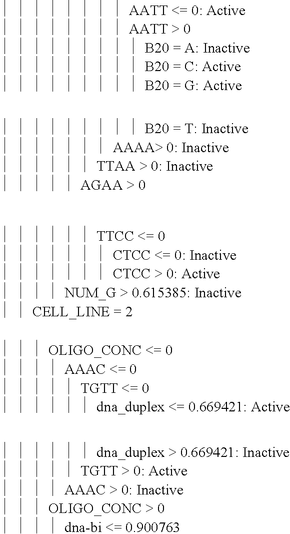

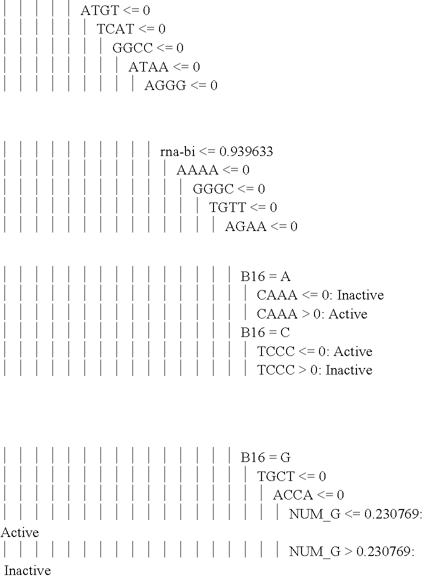

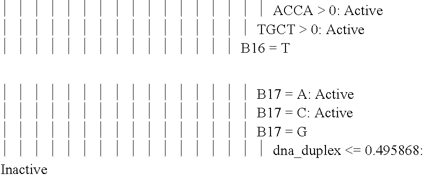

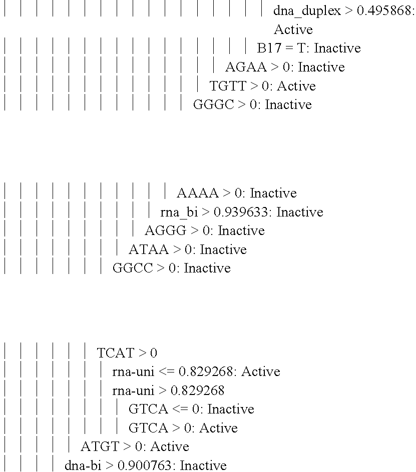

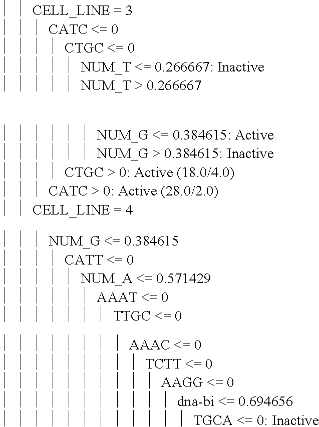

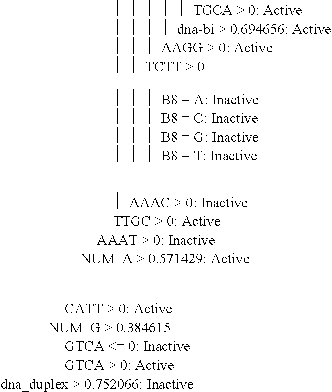

To make the model more readable, we generated a pruned form that displays less details:

TABLE 1.4

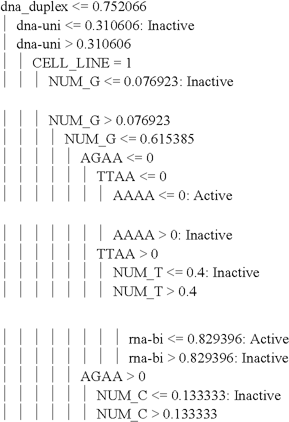

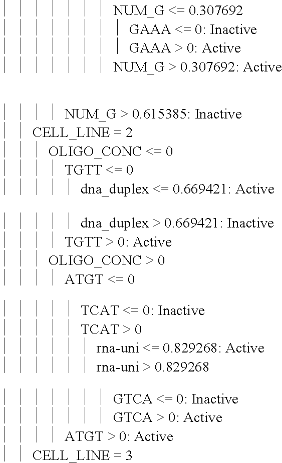

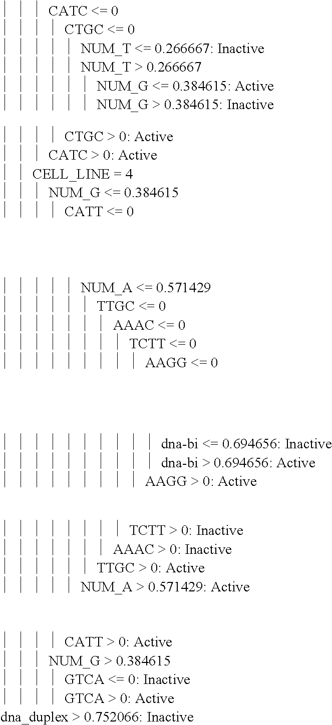

### Example 2

### Using ‘Flex’ Motifs in Predictive Modeling of Antisense Oligonucleotides

In the previous Example is presented an approach that included the energies as well as motifs, in addition to several other descriptors that helped build a more efficient predictive model of oligo activity. Moreover, a decision tree induction model that gives a human-readable output in the form of a hierarchical tree. This example evaluated to predicting 66% of correctly classified oligos, tested using 10-fold cross-validation.

A tetramotif is a four NT long subsequence in an antisense oligo sequence. The motif analysis of Isis Pharmaceuticals' data gave a list of more than fifty motifs that are positively and negatively related to oligo activity. We used this list of motifs as a part of the input into the decision tree learning schema to help us build a predictive model. There were a total of 88 attributes that were input to the model.

Reduction of attribute space, provided the predictive ability of the subset of attributes is at least as much as of the whole set, is always a good idea. The chance of the learning method getting ‘overwhelmed’ with the number of attributes can decrease, and often the predictive ability of the models produced with the reduced attribute set could increase. In this example, the 55 motifs were reduced to a smaller subset of attributes. The inherent noise in the dataset compelled the use of more flexible motifs rather than the fixed tetramers, as seen in this example.

Tetramers with ambiguity codes (Table 2.1) in certain locations, instead of only A's, C's, T's or G's. For example, TYYC would allow C or T in the second and third location, a T in the first, and a C in the fourth. In order to preserve the predictive ability of fixed motifs, a minimal outer cover of the motifs was determined. Following is a list of flex motifs found to be positively or negatively correlated to activity.

### List of Positive and Negative Flex Motifs

YCAT

CATB

TYYC

YCTG

WCCW

YTGC

MTGT

TGCW

TGTY

CTCY

GTCM

WWWW

AAAN

NAAA

GGSS

GRRG

AAGD

AGGS

ASAA

GCMG

TAAR

TKAA

TYTT

TABLE 2.1
Ambiguity codes

| IUPAC Code | Meaning | Complement |
|---|---|---|
| A | A | T |
| C | C | G |
| G | G | C |
| T/U | T | A |
| M | A or C | K |
| R | A or G | Y |
| W | A or T | W |
| S | C or G | S |
| Y | C or T | R |
| K | G or T | M |
| V | A or C or G | B |
| H | A or C or T | D |
| D | A or G or T | H |
| B | C or G or T | V |
| N | G or A or T or C | N |

This Example continues using the decision tree induction method. After adding the new flex motif attributes to the dataset a variety of experiments were performed searching for an optimal model by varying the architecture and list of parameters. The input to the decision tree induction method consisted of: oligo sequence information, flex motifs, free energy (ΔG) scores, cell line and concentration values.

Moreover, artificial attributes were introduced: dna_selfOligo, rna_selfOligo, ave_uni, ave_bi and selfOligo. Sometimes, an artificial attribute, such as an average or a sum of several values has more predictive power than the individual attributes. The dna_uni and dna_bi values were averaged to get the dna_selfOligo and rna_uni and rna_bi to calculate rna_selfOligo. The dna_uni and rna_uni, and dna_bi and rna_bi were also averaged to calculate ave_uni and ave_bi respectively. selfOligo score was calculated as an average of all four individual oligo scores. Also added was the sum of the occurrence of positive (POSflex) and negative motifs (NEGflex), and the difference of the two sums as well (POSf-NEGf), to help express occurrence of any kind of positive or negative motif, as well as the difference in oligos. Moreover, the Purine and Pyramidine scores, as well as the difference of the two (Purine=NUM_A+NUM_G, Pyramidine=NUM_T+NUM_C) was created.

The best performing model evaluated with 66.63% correctly classified instances, which was calculated using 10-fold evaluation method. This is slightly more than the result of the previous Example, and the true positive rate was increased by 2.5% as well. Following are the detailed evaluation results:

TABLE 2.2
Detailed Accuracy by Class

| TP Rate | FP Rate | Precision | Recall | F-Measure | Class |
|---|---|---|---|---|---|
| 66.8% | 33.5% | 60.8% | 66.8% | 63.6% | Active |
| 66.5% | 33.2% | 72.1% | 66.5% | 69.2% | Inactive |

TABLE 2.3
Confusion Matrix

| Active | Inactive | ← | classified as |
|---|---|---|---|
| 1675 | 834 | \| | Active |
| 1082 | 2150 | \| | Inactive |

TABLE 2.4

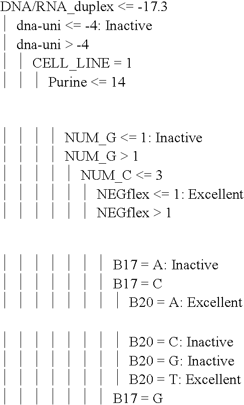

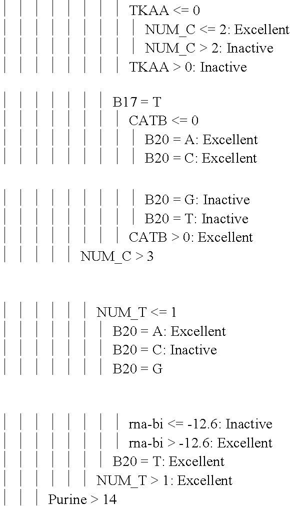

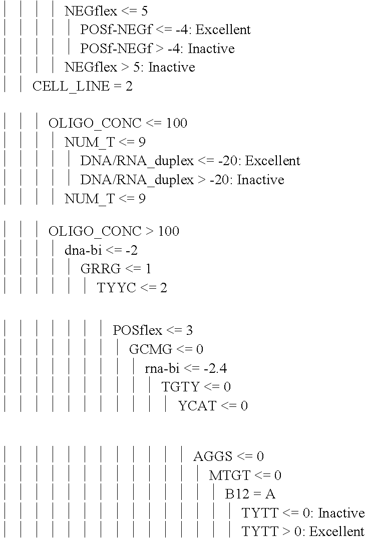

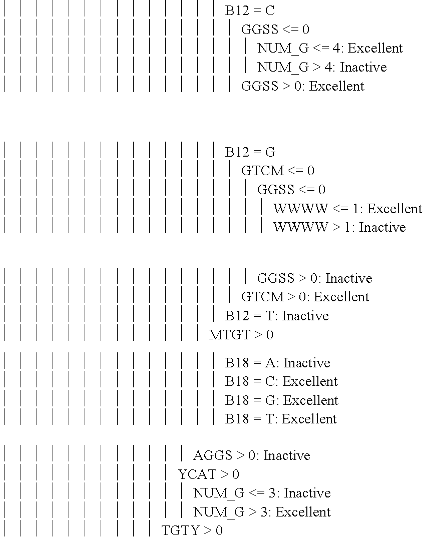

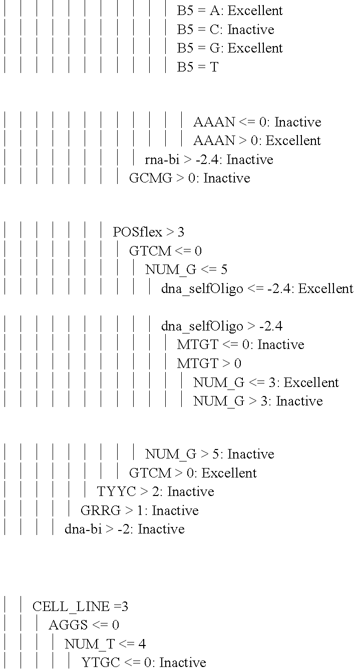

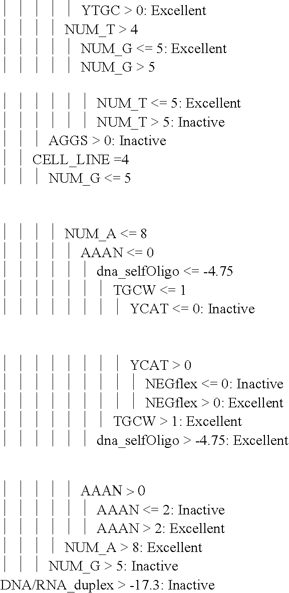

The use of flex motifs and artificial attributes helped the model overcome some of the noise and complexity in data and resulted in the increased model performance.

### Example 3

### The Relevance of Features in Predictive Modeling of Antisense Oligonucleotides

This Example incorporates Features into the logic used in previous Examples.

The features included exon, intron, start, stop, 3″UTR, 5″UTR and others (FIG. 1). An algorithm was devised for scoring the oligos based on whether they are designed to overlap a feature. The algorithm is feature-length dependent, and basically reflects the number of bases that overlap with the feature. Following is the list of features used:

TABLE 3.1
The list of DNA Structural Features Used
in Predictive Modeling of Oligo Activity

CDS
start
stop
transcriptional start
5′UTR
3′UTR
exon
intron
exon:exon junction
exon:intron junction
polyA signal

After adding the new features attributes to the dataset, a variety of experiments were performed searching for an optimal model by varying the architecture and list of parameters. The input to the decision tree induction method consisted of: oligo sequence information, flex motifs, free energy (DeltaG) scores, cell line and concentration, and the feature attributes.

The results are following. The best performing model evaluated with 70.21% correctly classified instances, which was calculated using 10-fold evaluation method. The evaluation score is 3.5% higher than the result of previous examples, with a higher true positive rate, and an increase of 6% of the true negative rate. Following are the detailed evaluation results:

TABLE 3.2
Detailed Accuracy by Class

| TP Rate | FP Rate | Precision | Recall | F-Measure | Class |
|---|---|---|---|---|---|
| 67.2% | 27.5% | 65.5% | 67.2% | 66.4% | Active |
| 72.5% | 32.8% | 74% | 72.5% | 73.3% | Inactive |

TABLE 3.3
Confusion Matrix

| Active | Inactive | ← | classified as |
|---|---|---|---|
| 1687 | 822 | \| | Active |
| 888 | 2344 | \| | Inactive |

TABLE 3.4
Predictive Model of Antisense Oligo Activity

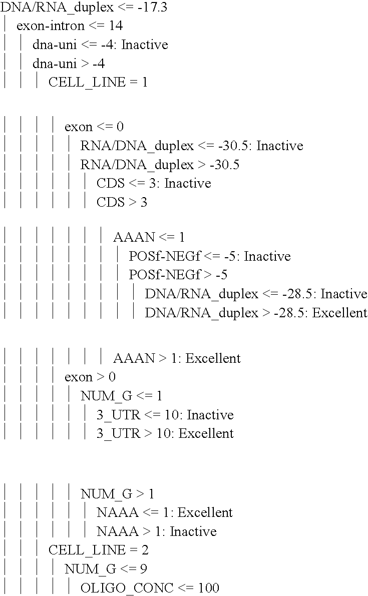

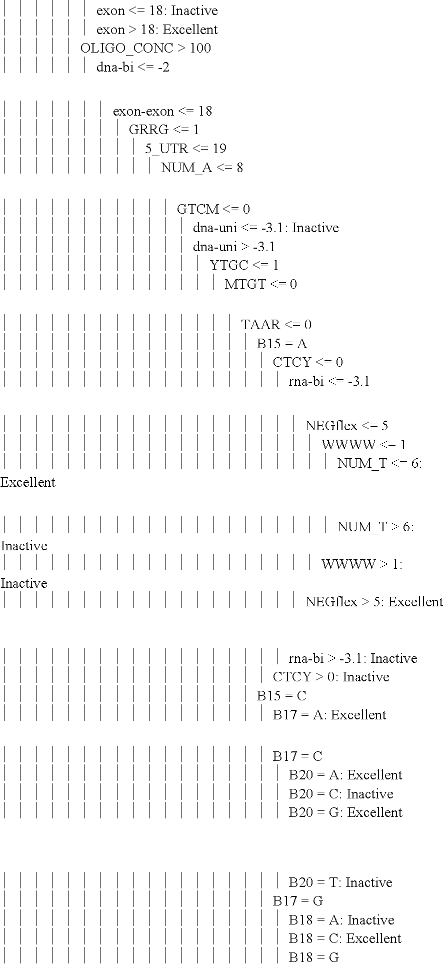

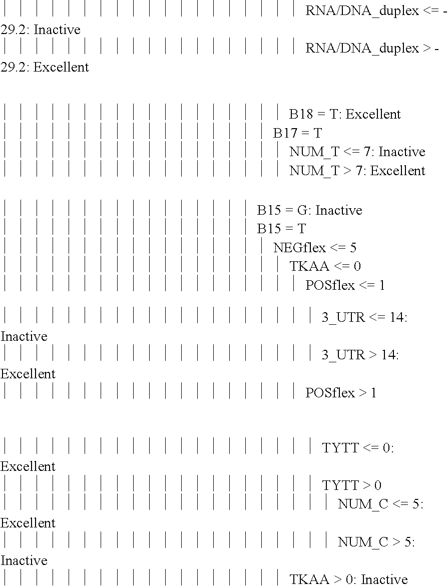

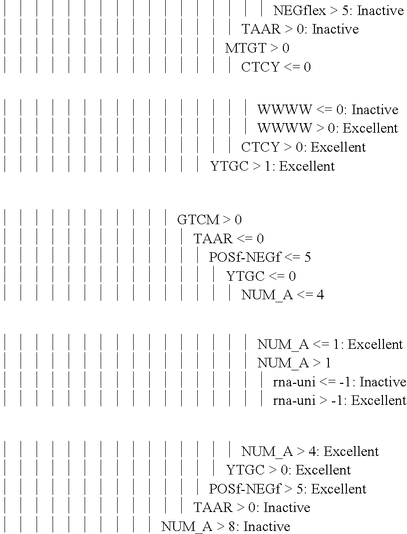

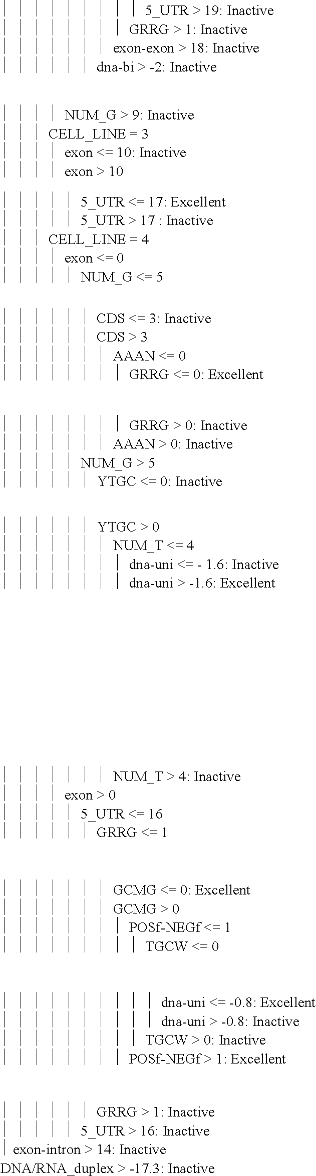

The use of features as descriptors may provide some benefit to help the model overcome some of the noise and complexity in real data; resulting in increased model performance and slightly better true positive and better true negative rates.

### Example 4

### mRNA Structure Information in Predictive Modeling of Antisense Oligonucleotides

This Example is directed to the incorporation of target structural information into the predictive paradigm. Two different types of scores: mFold and Pipas McMahon scores (Pipas and McMahon, 1975) were selected for use. The scores are different estimations of the mRNA structure. We added two mFold scores of two different regions around the oligo, as well as the P+M score calculated based on the revised Pipas and McMahon algorithm.

This Example continues to use the decision tree induction method. The input to the decision tree induction method consisted of: oligo sequence information, flex motifs, free energy (ΔG) scores, cell line and concentration, the feature attributes and the new mRNA structure attributes.

The results are following. The best performing model evaluated with 71.2419%

correctly classified instances, which was calculated using 10-fold evaluation method.

TABLE 4.1
Detailed Accuracy by Class

| TP Rate | FP Rate | Precision | Recall | F-Measure | Class |
|---|---|---|---|---|---|
| 68.8% | 26.9% | 66.5% | 68.8% | 67.7% | Active |
| 73.1% | 31.2% | 75.1% | 73.1% | 74.1% | Inactive |

TABLE 4.2
Confusion Matrix

| Active | Inactive | ← | classified as |
|---|---|---|---|
| 1727 | 782 | \| | Active |
| 869 | 2363 | \| | Inactive |

TABLE 4.3
Predictive Model of Antisense Oligo Activity

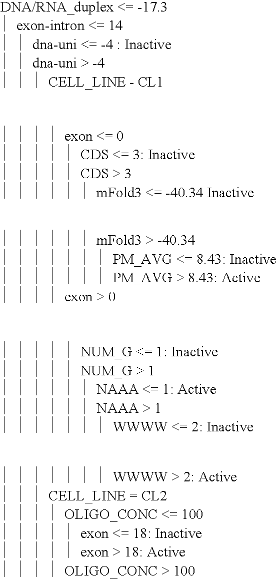

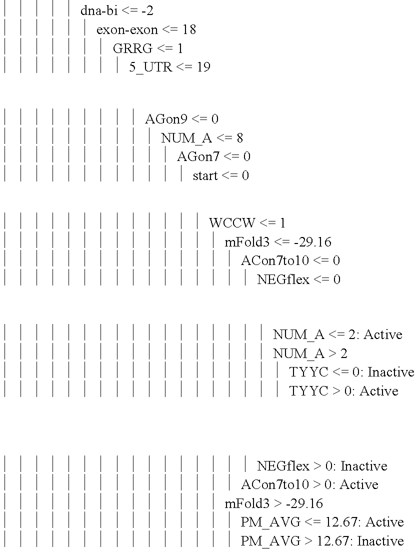

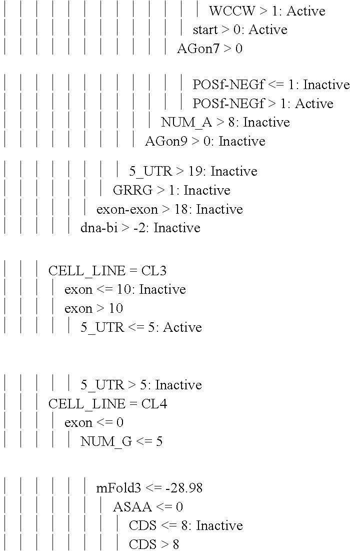

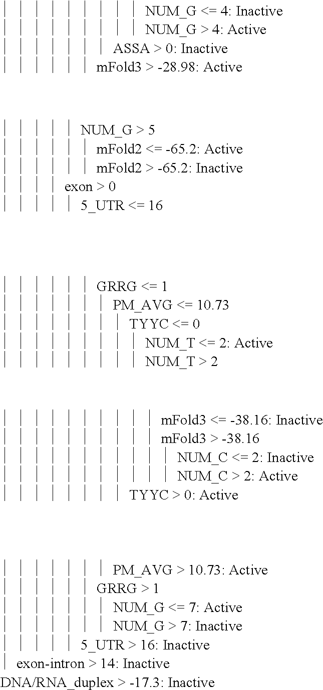

The use of mRNA structural information as descriptors may help the model overcome some of the noise and complexity in data thereby result in increased model performance.

### Example 5

### RNAse H Motifs in Predictive Modeling of Antisense Oligonucleotides

This Example is directed to the incorporation of certain RNAse H preferred cleaving sites, around the middle of the oligo into the predictive algorithm. The RNA dimers hypothesized to be good are GU, CU and UG. This translates to AC or AG or CA starting at positions 7-10 in the oligo. These sites were termed favorable motifs RNAse H motifs.

The attributes added were: ACon7 ACon8, ACon9, ACon10, AGon7, AGon8, AGon9, AGon10, CAon7, CAon8, CAon9, CAon10. We also added ACon7to10, AGon7to10 and CAon7to10 as the sums of appropriate single motif occurrences, as well as RNase H that counts the number of any of the RNase H motifs starting at any of the positions (7, 8, 9, or 10) in a single oligo.

This model evaluated with 71.6948% correctly classified instances, which was calculated using 10-fold evaluation method. Following are the detailed evaluation results:

TABLE 5.1
Detailed Accuracy by Class

| TP Rate | FP Rate | Precision | Recall | F-Measure | Class |
|---|---|---|---|---|---|
| 68.9% | 26.1% | 67.2% | 68.9% | 68.0% | Active |
| 73.9% | 31.1% | 75.4% | 73.4% | 74.6% | Inactive |

TABLE 5.2
Confusion Matrix

| Active | Inactive | ← | classified as |
|---|---|---|---|
| 1728 | 781 | \| | Active |
| 844 | 2388 | \| | Inactive |

TABLE 5.3
Predictive Model of Anlisense Oligo Activity

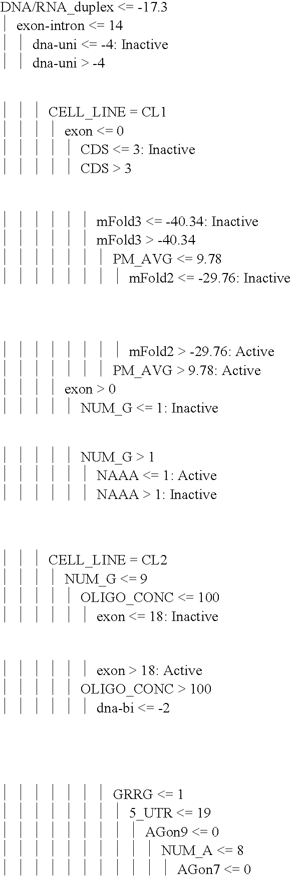

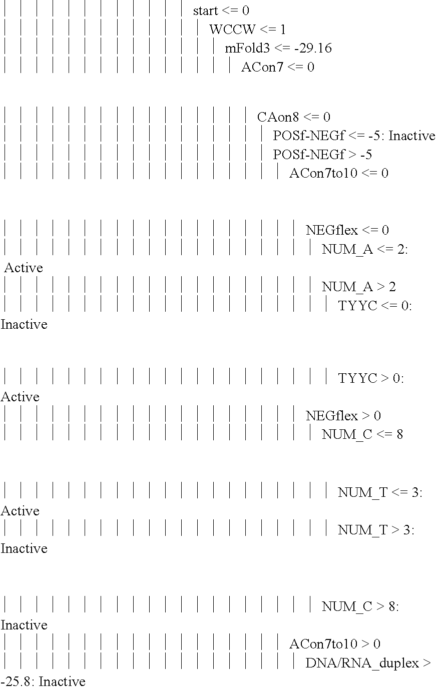

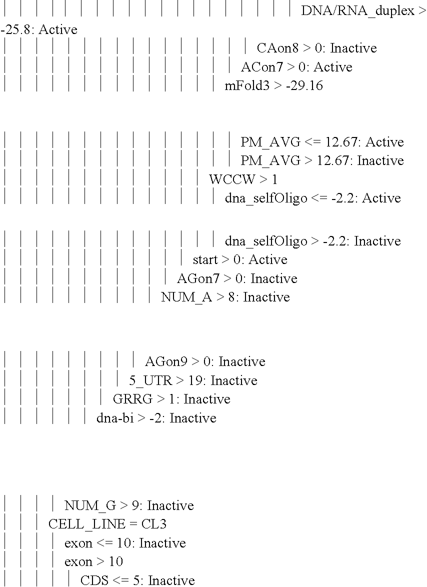

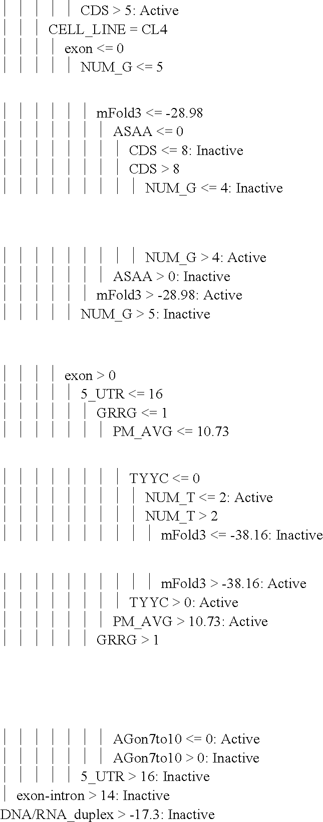

### Example 6

### Amplicon Information in Predictive Modeling of Antisense Oligonucleotides

In this Example the amplicon information was added to the dataset. Amplicon oligos are oligos that lie in between the forward and reverse primer of the primer probe set Amplicon oligos or amplicons for short can be active or inactive. Active amplicons can be false positives and should only be judicially incorporated into any dataset.

Several datasets were tested: the current dataset with the amplicon attribute added (=1 if oligo is an amplicon, =0 otherwise), a dataset with all the amplicon oligos excluded, as well as a dataset where only inactive amplicon oligos were kept, and active ones were excluded.

This model evaluated with 73.7032% correctly classified instances, which was calculated using 10-fold evaluation method.

TABLE 6.1
Detailed Accuracy by Class

| TP Rate | FP Rate | Precision | Recall | F-Measure | Class |
|---|---|---|---|---|---|
| 62.9% | 19.5% | 66.9% | 62.9% | 64.9% | Active |
| 80.5% | 37.1% | 77.5% | 80.5% | 79.0% | Inactive |

FIG. 6.2 Confusion Matrix

| Active | Inactive | ← | classified as |
|---|---|---|---|
| 1278 | 753 | \| | Active |
| 631 | 2601 | \| | Inactive |

TABLE 6.3
Predictive Model of Antisense Oligo Activity

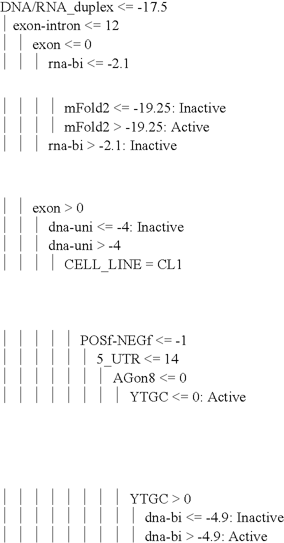

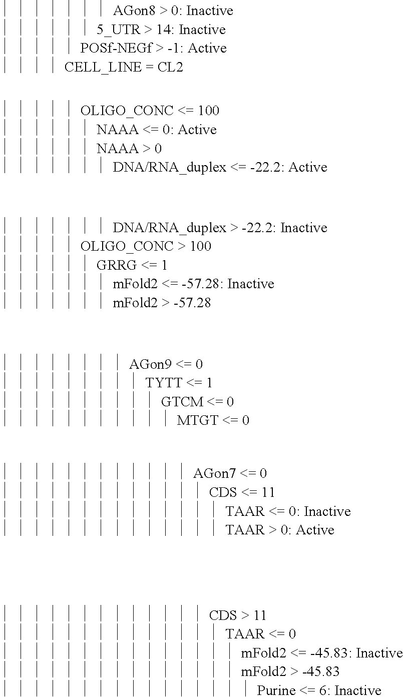

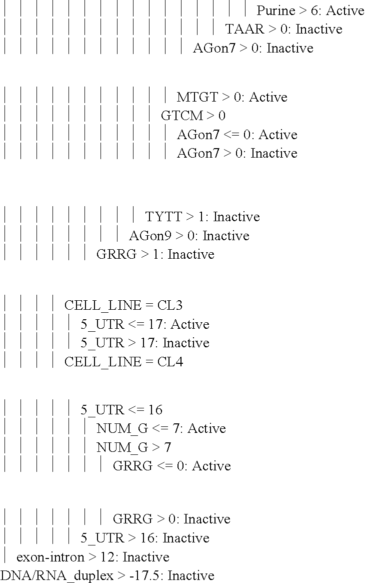

### Example 7

### Comparison of Different Data Mining Methods in Predictive Modeling of Antisense Oligonucleotides

This Example is directed to the types of predictive paradigm available. Antisense oligonucleotides have been used to inhibit the expression of genes involved in various diseases. Several methods have been tested in efforts to predict the activity of an antisense oligonucleotide, ranging from simple statistical methods to various data mining and machine learning methods. For example, in previous work (Fu et al, 1998, Matveeva et al; 2000, Giddings et al, 2002) revealed a correlation between the short sequence motifs (tetramotifs or shorter) as well as certain ΔG energy scores (Matveeva et al, 2001) and antisense oligo activity using logistic regression and simple T tests. Giddings et al (NAR 2002) presented an artificial neural network model that takes forty tetramotifs as input, and outputs a predictive level of activity. The model evaluated to predicting 53% of correctly classified instances using cross-validation. A decision tree induction method was to learn and produce a human-readable output in the form of a hierarchical tree. This model evaluated to predicting 72% of correctly classified instances, tested using 10-fold cross-validation, which compared to state-of-the-art model in the literature (Giddings et al, NAR 2002).

In this example is presented the use of different data mining methods and schemas in building predictive models of oligo activity. Once a majority of the attributes describing an antisense oligonucleotide have been collected, representatives of a variety of learning method types must be considered. Since the activity of an oligo can be represented both as a discrete and a continuous value, using nominal as well as numeric prediction algorithms must also be considered. Regression tree induction, decision tree induction, clustering, neural network methods and multi-variate regression tree induction method are among the predictive algorithms tested.

### Decision Trees

Decision tree learning is one of the most popular and practical methods for inductive inference. It is a method for approximating discrete-valued functions, where a decision tree represents the learned function. Decision tree induction is robust to noisy data and capable of learning disjunctive expressions. Decision trees are capable of handling training examples with missing attribute values and attributes with different costs. This algorithm has been successfully applied to a wide range of learning tasks, from medical diagnosis to classifying equipment malfunctions by cause (Mitchell, 1997).

Decision trees classify instances by sorting them down the tree from the root to some leaf node, which provides the classification of the instance. Each node in the tree specifies a test of some attribute of the instance, and each branch descending from that node corresponds to one of the possible values for this attribute.

### Regression Trees

Regression trees are a type of decision trees that deal with continuous variables. Regression trees are non-parametric models, an advantage of which is a high computational efficiency and a good compromise between comprehensibility and predictive accuracy. The regression tree method can be applied to very large datasets in which only a small proportion of the predictors are valuable for classification.

The task of a regression method is to obtain a model from a sample of objects belonging to an unknown regression function (Torgo, 1999). These methods perform induction by means of an efficient recursive-partitioning algorithm. As with decision tree induction, one decision that needs to be made during the tree growth is how to choose the best split for each node. This task is made more complicated by the presence of continuous variables. This task may also be understood as a means of incorporating influence indicators in the dataset. These indicators provide additional information relative to the associated object or parameter and that objects quantum of influence on activity.

### Clustering

Clustering is a machine learning method that uses unsupervised learning. A clustering algorithm partitions input instances into a fixed number of subsets or clusters so that the inputs in the same cluster are dose to one another with respect to some specified metric (Dean et al, 1995). This technique can easily predict both categorical and nominal data.

There are several different clustering methods. We have used and tested the classic k-means algorithm (McQueen, 1967), which is a simple straightforward technique that forms clusters in numeric domains, by partitioning instances into disjoint clusters, the expectation-minimization (EM) algorithm, as well as hierarchical clustering methods: EM is similar to the k-means method in that it first elects cluster parameters, starts with the initial guesses of the parameters, calculates cluster probabilities and iterates while adjusting cluster probabilities of the instances in each iteration. Hierarchical clustering operates incrementally on input data-instance by instance to form concept hierarchies. It does not have a predefined number of clusters. A hierarchical method (e.g. COBWEB) grows a tree starting at an empty root node, adding instances one by one, and updating the tree accordingly, as determined by a probabilistic measure called the category utility.

### Artificial Neural Networks (ANN)

Historically, some ANNs were inspired and modeled based on biological neural nets, especially the parallel architecture of animal brains in order to produce intelligent “brain like” performing systems. Neural networks can be described as a form of multiprocessor computer system, with simple processing elements, a high degree of interconnection, simple scalar messages, and adaptive interaction between elements (Smith, 1996).

An ANN is a network of many simple units, which could possibly have a small amount of local memory, connected by communication channels capable of carrying numeric data of various kinds. These units operate only locally on the data they receive through their inputs. The processing ability of the network is stored in the inter-unit connection strength or weights that are being adapted based on a set of training data. Most ANNs have a training rule whose role is to adjust weights of connections based on the input data. They are capable of learning from experience and generalizing beyond the training data (Sarle, 2001).

There are many different kinds of neural networks, including those that learn in a supervised or unsupervised fashion, and those that have a feed-forward or feedback topology. In supervised learning, the neural net is provided with the correct result of target values during the training, while in unsupervised, it is not. Feed-forward propagation network has a flow of information through a neural net from its input to its output layer. A back-propagation algorithm is mainly used by multi-layer-perceptrons to change the weights connecting the network's input, hidden and output layers. This algorithm uses a forward propagation to determine the output error in order to change the weight values in the backward direction. Most practical application of neural nets fall under the supervised learning feedback type of ANN.

We ran a variety of experiments and tests and concluded that using decision trees is the most beneficial in building predictive models of oligo activity. First, decision trees are able to handle noise and missing attributes exceptionally. Second, the models are comprehensive and offer scientific insight into the importance of various data descriptors. Third, decision trees allow for various levels of generalization—we can build a very specific, highly detailed model, we can generalize, or grossly generalize and look at the data from a very high perspective. Fourthly, they produced the higher 10-fold evaluation scores that estimate the performance of the model on unseen data. Further, decision trees allow the model trees to be pruned using scientific expertise, for the leaves to have a certain minimum number of instances, tailored towards the specifics of the dataset, and they can handle large amounts of noise, so highly characteristic of scientific datasets. When the models are human-readable and represented in a nice form of a tree, they can be combined with alike models as well as models built using different methods. We found decision tree induction to be the most useful method in predictive modeling of Antisense oligonucleotide activity.

In Table 7.1 is a summary of the described analysis. The quality of produced model, their evaluation, size of the model, relative ease of training, training time, interpretability and comprehensibility of the model were considered.

TABLE 7.1
Comparative Study of the Data Mining Methods

| | | | | | | Interpretability |
|---|---|---|---|---|---|---|
| | Produced | | Size of the | Ease of | Training | and |
| | Models | Evaluation | model | training | time | Comprehensibility |
| Regression | Very | Correlation | 40-1000 | Easy to | Moderate | Easy |
| Trees | Good | coefficient | leaves | moderate | | |
| | | 0.5 | | | | |
| Clustering | Poor | N/A | 10 clusters | Easy | Short | Moderate |
| Hierarchical | Good | N/A | 130 | Moderate | Moderate | Moderate |
| Clustering | | | clusters | | | |
| Neural | Very | 68% | 200 × 100 × | Moderate to | Lengthy | Difficult |
| Networks | Good to | correctly | 50 × 30 × 2 | difficult | | |
| | Excellent | classified | matrix | | | |
| | | instances | | | | |
| | | (10-fold) | | | | |
| Decision | Excellent | 74% | 50-500 | Moderate | Moderate | Easy |
| Trees | | correctly | leaves | | | |
| | | classified | | | | |
| | | instances | | | | |
| | | (10-fold) | | | | |

### Example 8

Here we report the efforts to create a predictive model that would perform better in predicting Active antisense oligonucleotides as compared previously reported models. We use a predictive hybrid model of oligonucleotide activity that includes individual models built on different subsets or clusters of data. We also use different data mining methods, as they have different characteristics, and as we anticipated, would be better in overcoming the various aspects of predictive modeling of our dataset.

An advantage of building a hybrid model is in choosing the best algorithm to describe and predict various clusters of our data, as well as the whole dataset, by concentrating on a slightly different aspect of the data with the use of another technique. The hybrid model we built is tailored to the complexities of our dataset. Combining various data mining methods allowed us to use all of their advantages without having to deal with any of the restrictions. The hybrid model consists of the best performing predictive models on each of the entire collection of prevalent clusters of our dataset, which are then combined using an algorithm to assign situation-dependent priorities into the Hybrid Model.

We used a starting screening data that underwent thorough cleaning and filtering to reduce the amount of noise in the dataset. We then kept only highly Active and highly Inactive oligos. We called this Dataset 1. We also used the initial dataset and excluded the Active amplicon oligos, as amplicon oligos could possibly be false positives. This dataset was named Dataset 2

We used the following two data mining methods to build the submodels of our hybrid model: Decision Tree Induction and Neural Network learning.

Since the cell line and concentration information are not readily available to the scientists until right before the screen we decided to force-feed the cell line information by providing the two combinations of cell line per a species (or one in case of the Rat species) as shown in Table 8.

TABLE 8
The Cell Line Combinations for Each Species

| | CELL_LINE_1 | CELL_LINE_2 |
|---|---|---|
| Human | A549 | T-24 |
| Mouse | 3T3-L1 undifferentiated | b.END |
| Rat | A10 | A10 |

We decided to incorporate the best retrained Decision Tree model build on the dataset containing only Inactive Amplicons (Dataset 1), as well as the Excellent and some Inactives dataset (Dataset 2). We also included a Neural Network built on only Inactive Amplicons dataset. Each of these models was built using cell line 1 and then cell line 2 information. We created a hybrid DT model for each cell line, followed by the hybrid model consisting of the two DT models and the NN model.

The best predictive scores in predicting Actives were obtained when at least one of the hybrid models for one or the other cell line was predicting an Active oligo. Similarly, the best predictive scores in predicting Inactives were obtained when at least one of the hybrid models for one or the other cell line was predicting an Inactive oligo. We used this information to design an algorithm that would create a Final Hybrid Predictive Model by combining the two different-cell-line hybrid models.

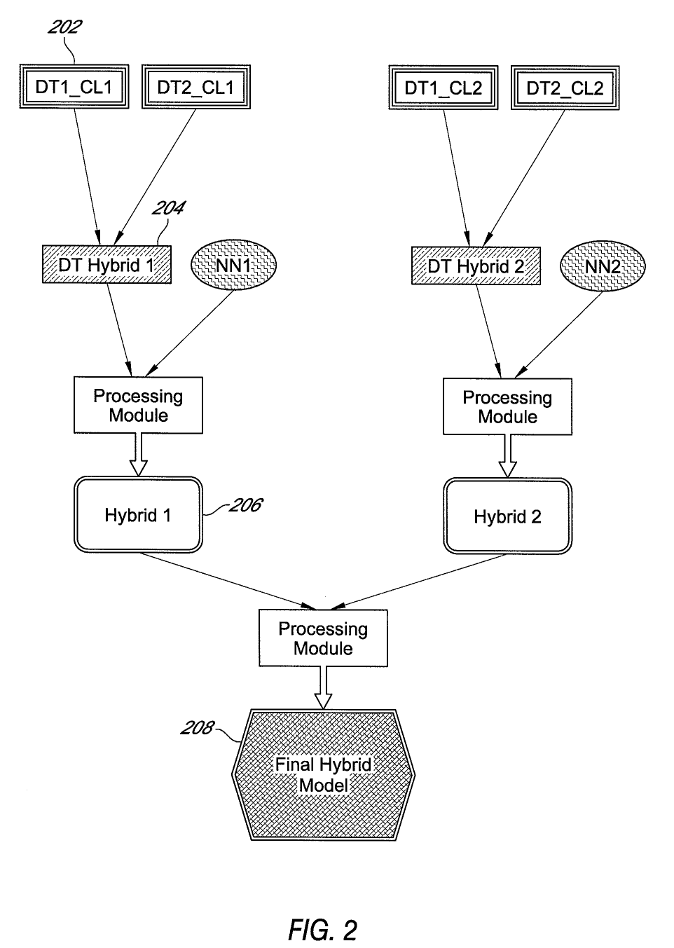

The Final Hybrid model evaluated to correctly predicting 70.95% of Active oligos, 75.9231% of Active oligos when predictive Okays (since they are not Inactive oligos) were calculated into the score, and 84.9319% of Inactive oligos. Combined scores give 78% or 80.4% (with Okays) of correctly classified instances. Compared to the state of the art model in the literature (Giddings et al, 2002), this result is an increase of 47% or 52% (with Okays) in model performance. FIG. 2 illustrates the architecture of the Hybrid Model. ‘DT1_CL1’ 202 stands for the Decision Tree model built on Dataset 1 for the cell line 1. ‘DT Hybrid1’ 204 stands for the hybrid DT model for cell line 1. ‘Hybrid1’ 206 represents the Hybrid model built for cell line 1, while ‘Final Hybrid’ 208 stands for the all-cell-line Final Predictive Hybrid model. In the Processing Modules, the two scores are combined, and then a list of priority rules is applied. For example, if at least one of the scores is Active, the outcome is proclaimed Active. If the confidence factor of a prediction being active is low (i.e. less than 0.2), the outcome is pronounced ‘Okay.’

1-18. (canceled)

19. A method for selecting a preferred set of oligonucleotides complementary to a target comprising:
generating a test-set comprising a plurality of oligonucleotides complementary to a target;
generating a training-set database comprising a plurality of oligonucleotides known to be active and a plurality of oligonucleotides known to be inactive and a plurality of parameters associated with each oligonucleotide;
generating a first and a second predictive model by from the training-set database; and
applying the first and second predictive model to the test-set and thereby selecting a preferred set of oligonucleotides complementary to a target.

20. The method of claim 19, wherein the first predictive model is a decision tree model.

21. The method of claim 19, wherein the first predictive model is a neural network model.

22. The method of claim 19, wherein the first predictive model is a clustering model.

23. The method of claim 19, wherein the first predictive model is a hierarchical clustering model.

24. The method of claim 19, wherein the first predictive model is a regression tree model.

25. The method of claim 20, wherein the second predictive model is a decision tree model.

26. The method of claim 20, wherein the second predictive model is a neural network model.

27. The method of claim 20, wherein the second predictive model is a clustering model.

28. The method of claim 20, wherein the second predictive model is a hierarchical clustering model.

29. The method of claim 20, wherein the second predictive model is a regression tree model.

30. A method for selecting a preferred set of oligonucleotides complementary to a target comprising:
generating a test-set comprising a plurality of oligonucleotides complementary to a target;
generating a training-set database comprising a plurality of oligonucleotides known to be active and a plurality of oligonucleotides known to be inactive and a plurality of parameters associated with each oligonucleotide;
generating a decision tree predictive model by from the training-set database; and
applying the decision tree predictive model to the test-set and thereby selecting a preferred set of oligonucleotides complementary to a target.

31. A method for selecting a preferred set of oligonucleotides complementary to a target comprising:
generating a test-set comprising a plurality of oligonucleotides complementary to a target;
generating a training-set database comprising a plurality of oligonucleotides known to be active and a plurality of oligonucleotides known to be inactive and a plurality of parameters associated with each oligonucleotide;
generating a neural network predictive model by from the training-set database; and
applying the neural network predictive model to the test-set and thereby selecting a preferred set of oligonucleotides complementary to a target.

32. A method for selecting a preferred set of oligonucleotides complementary to a target comprising:
generating a test-set comprising a plurality of oligonucleotides complementary to a target;
generating a first and a second training-set database wherein each of the first and the second training-set database independently comprises a plurality of oligonucleotides known to be active and a plurality of oligonucleotides known to be inactive and a plurality of parameters associated with each oligonucleotide;
generating a first predictive model by from the first training-set database and a second predictive model from the second training-set database; and
separately applying the first and the second predictive models to the test-set and thereby selecting a preferred set of oligonucleotides complementary to a target.

33. The method of claim 32, wherein the first and second training set comprise data for the same oligonucleotides tested in different cell types.

34. The method of claim 32, wherein each of the first and second predictive model is a decision tree model.
